(19) United States
(12) Patent Application Publication
(10) Pub. No.: US 2019/0216726 A1
(43) Pub. Date: Jul. 18, 2019

(54) Particles, Compositions and Methods for Ophthalmic and/or Other Applications

(71) Applicant: Kala Pharmaceuticals, Inc., Watertown, MA (US)

(72) Inventors: Winston Zapanta ONG, Watertown, MA (US); Minh Ngoc NGUYEN, Watertown, MA (US)

(21) Appl. No.: 16/331,474

(22) PCT Filed: Sep. 15, 2017
(86) PCT No.: PCT/US2017/051869

§ 371 Date: Mar. 7, 2019

Related U.S. Application Data
(60) Provisional application No. 62/395,984, filed on Sep. 16, 2016.

(51) Int. Cl. A61K 9/00; A61K 9/50; A61K 31/565; A61K 31/58

(52) U.S. Cl. CPC A61K 9/0048; A61K 9/5015; A61K 31/58; A61K 9/5031; A61K 31/565; A61K 9/5026

(57) ABSTRACT

This disclosure relates to particles, compositions, and methods that aid particle transport in mucus are provided. The particles, compositions, and methods may be used, in some instances, for ophthalmic and/or other applications.

### CROSS-REFERENCE TO RELATED APPLICATIONS

The present application claims the benefit under to U.S. Provisional Patent Application 62/395,984 filed Sep. 16, 2016, the entire contents of which are incorporated by reference herein.

### FIELD

The present disclosure generally relates to particles, compositions, and methods that aid particle transport in mucus. The particles, compositions, and methods may be used in ophthalmic and/or other applications.

### BACKGROUND

A mucus layer present at various points of entry into the body, including the eyes, nose, lungs, gastrointestinal tract, and female reproductive tract, is naturally adhesive and serves to protect the body against pathogens, allergens, and debris by effectively trapping and quickly removing them via mucus turnover. For effective delivery of therapeutic, diagnostic, or imaging particles via mucus membranes, the particles must be able to readily penetrate the mucus layer to avoid mucus adhesion and rapid mucus clearance.

Particles (including microparticles and nanoparticles) that incorporate pharmaceutical agents are particularly useful for ophthalmic applications. However, often it is difficult for administered particles to be delivered to an eye tissue in effective amounts due to rapid clearance and/or other reasons. Accordingly, new methods and compositions for administration (e.g., topical application or direct injection) of pharmaceutical agents to the eye would be beneficial.

### SUMMARY

Disclosed herein are pharmaceutical compositions comprising mucus-penetrating particles containing hydrocortisone (4-pregenen-11β-17-21-triol-3,20-dione) derivatives. In certain embodiments, the derivative is:

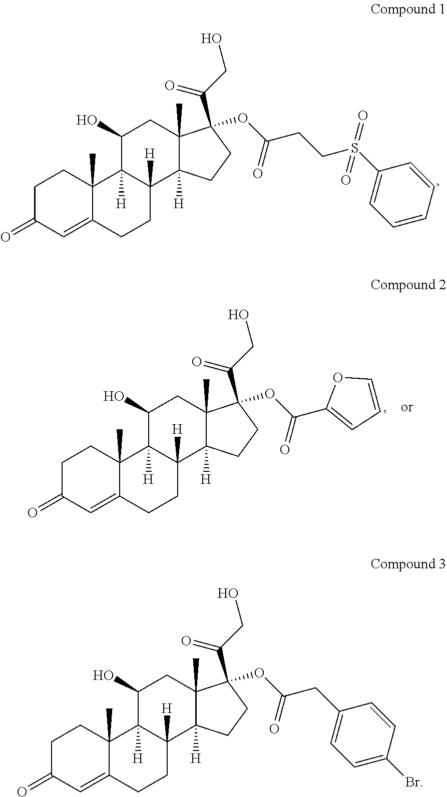

In some embodiments, the hydrocortisone derivative is (10R,11S,13S,17R)-11-hydroxy-17-(2-hydroxyacetyl)-10,13-dimethyl-3-oxo-2,3,6,7,8,9,10,11,12,13,14,15,16,17-tetradecahydro-1H-cyclopenta[a]phenanthren-17-yl 3-(phenylsulfonyl)propanoate (Compound 1), (10R,11S,13S,17R)-11-hydroxy-17-(2-hydroxyacetyl)-10,13-dimethyl-3-oxo-2,3,6,7,8,9,10,11,12,13,14,15,16,17-tetradecahydro-1H-cyclopenta[a]phenanthren-17-yl furan-2-carboxylate (Compound 2), or (10R,11S,13S,17R)-11-hydroxy-17-(2-hydroxyacetyl)-10,13-dimethyl-3-oxo-2,3,6,7,8,9,10,11,12,13,14,15,16,17-tetradecahydro-1H-cyclopenta[a]phenanthren-17-yl 2-(4-bromophenyl)acetate (Compound 3).

Some embodiments include a pharmaceutical composition suitable for administration to an eye, comprising: a plurality of coated particles, comprising a core particle comprising a hydrocortisone derivative selected from Compounds 1, 2, and 3; a mucus penetration-enhancing coating comprising a surface-altering agent surrounding the core particle, wherein the surface-altering agent comprises: a) a triblock copolymer comprising a hydrophilic block-hydrophobic block-hydrophilic block configuration, wherein the hydrophobic block has a molecular weight of at least about 2 kDa, and the hydrophilic blocks constitute at least about 15 wt % of the triblock copolymer, the hydrophobic block associates with the surface of the core particle, and the hydrophilic block is present at the surface of the coated particle and renders the coated particle hydrophilic, b) a synthetic polymer having pendant hydroxyl and ester groups in the backbone of the polymer, the polymer having a molecular weight of at least about 1 kDa and less than or equal to about 1000 kDa, wherein the polymer is at least about 30% hydrolyzed and less than about 95% hydrolyzed, or c) a polysorbate; wherein the surface altering agent is present on the outer surface of the core particle at a density of at least 0.01 molecules/nm², wherein the surface altering agent is present in the pharmaceutical composition in an amount of between about 0.001% to about 5% by weight; and an ophthalmically acceptable carrier, additive, or diluent.

Some embodiments include a pharmaceutical composition suitable for treating an ocular disorder by administration to an eye, comprising: a plurality of coated particles, comprising a core particle comprising a hydrocortisone derivative disclosed herein and a mucus penetration-enhancing coating comprising a surface-altering agent surrounding the core particle, wherein the surface-altering agent comprises: a) a triblock copolymer comprising a hydrophilic block-hydrophobic block-hydrophilic block configuration, wherein the hydrophobic block has a molecular weight of at least about 2 kDa, and the hydrophilic blocks constitute at least about 15 wt % of the triblock copolymer, b) a synthetic polymer having pendant hydroxyl groups on the backbone of the polymer, the polymer having a molecular weight of at least about 1 kDa and less than or equal to about 1000 kDa, wherein the polymer is at least about 30% hydrolyzed and less than about 95% hydrolyzed, or c) a polysorbate, wherein the plurality of coated particles have an average smallest cross-sectional dimension of less than about 1 micron; and wherein the coating on the core particle is present in a sufficient amount to increase the concentration of the hydrocortisone derivative in a cornea or an aqueous humor after administration to the eye, compared to the concentration of the hydrocortisone derivative in the cornea or the aqueous humor when administered as a core particle without the coating.

Also provided herein are methods of treating, diagnosing, preventing, or managing an ocular condition in a subject, the method comprising: administering a pharmaceutical composition described herein, such as a composition comprising a hydrocortisone derivative-containing mucus-penetrating particles to an eye of a subject and thereby delivering the hydrocortisone derivative to a tissue in the eye of the subject.

### BRIEF DESCRIPTION OF THE DRAWINGS

FIG. 1 is a schematic drawing of a mucus-penetrating particle having a coating and a core according to one set of embodiments.

FIG. 2A depicts a histogram showing the ensemble averaged velocity <Vₘₑₐₙ> in human cervicovaginal mucus (CVM) for 200 nm carboxylated polystyrene particles (PSCOO⁻; negative control), 200 nm PEGylated polystyrene particles (positive control), and nanoparticles (sample) made by milling and coated with different surface-altering agents according to one set of embodiments. FIG. 2B is a plot showing the relative velocity <Vₘₑₐₙ>ᵣₑₗ in CVM for nanoparticles made by milling and coated with different surface-altering agents according to one set of embodiments.

FIGS. 3A-3D are histograms showing distribution of trajectory-mean velocity Vₘₑₐₙ in CVM within an ensemble of nanoparticles coated with the surface-altering agents Pluronic® F127 (FIG. 3A), Pluronic® F87 (FIG. 3B), Pluronic® F108 (FIG. 3C), and Kollidon 25 (FIG. 3D) according to one set of embodiments.

FIG. 4 is a plot showing <Vₘₑₐₙ>ᵣₑₗ in CVM for nanoparticles coated with different poly(ethylene oxide)-poly(propylene oxide)-poly(ethylene oxide) (PEO-PPO-PEO) Pluronic® triblock copolymers, mapped with respect to molecular weight of the PPO block and the PEO weight content (%), according to one set of embodiments.

FIG. 5A is a histogram showing the ensemble averaged velocity <Vₘₑₐₙ> in human CVM for PSCOO⁻ particles coated with various poly(vinyl alcohols) (PVAs) according to one set of embodiments. FIG. 5B is a plot showing the relative velocity <Vₘₑₐₙ>ᵣₑₗ in CVM for PSCOO⁻ particles coated with various PVAs according to one set of embodiments.

FIG. 6 is a plot showing relative velocity <Vₘₑₐₙ>ᵣₑₗ in CVM for PSCOO⁻ particles incubated with various PVAs mapped according to the PVA's molecular weight and degree of hydrolysis, according to one set of embodiments. Each data point represents <Vₘₑₐₙ>ᵣₑₗ for the particles stabilized with a specific PVA.

FIGS. 7A-7B are plots showing bulk transport in CVM in vitro of PSCOO⁻ nanoparticles coated with various PVAs in two different CVM samples, according to one set of embodiments. Negative controls are uncoated 200 nm PSCOO⁻ particles; Positive controls are 200 nm PSCOO⁻ particles coated with Pluronic® F127.

FIGS. 8A-8B are plots showing ensemble-average velocity <Vₘₑₐₙ> (FIG. 8A) and relative sample velocity <Vₘₑₐₙ>ᵣₑₗ (FIG. 8B) for poly(lactic acid) (PLA) nanoparticles (sample) prepared by emulsification with various PVAs as measured by multiple-particle tracking in CVM, according to one set of embodiments.

FIGS. 9A-9B are plots showing ensemble-average velocity <Vₘₑₐₙ> (FIG. 9A) and relative sample velocity <Vₘₑₐₙ>ᵣₑₗ (FIG. 9B) for pyrene nanoparticles (sample) and controls as measured by multiple-particle tracking in CVM, according to one set of embodiments.

FIGS. 10A-10F are representative CVM velocity (Vₘₑₐₙ) distribution histograms for pyrene nanoparticles obtained with surface-altering agents PVA2K75 (FIG. 10A), PVA9K80 (FIG. 10B), PVA31K98 (FIG. 10C), PVA85K99 (FIG. 10D), Kollidon 25 (FIG. 10E), and Kollicoat IR (FIG. 10F) (SAMPLE=pyrene nanoparticles, POSITIVE=200 nm PS-PEG5K, NEGATIVE=200 nm PS-COO); according to one set of embodiments.

FIG. 11 is a plot of relative velocity <Vₘₑₐₙ>ᵣₑₗ for pyrene nanoparticles coated with PVA in CVM mapped according to the PVA's molecular weight and degree of hydrolysis according to one set of embodiments.

FIG. 12 is a bar graph showing the density of Pluronic® F127 on the surface of fluticasone propionate and loteprednol etabonate microparticles, according to one set of embodiments.

FIG. 13 is a plot showing the mass transport through CVM for solid particles having different core materials that are coated with either Pluronic® F127 (MPP, mucus-penetrating particles) or sodium dodecyl sulfate (CP, conventional particles, a negative control), according to one set of embodiments.

FIG. 14 depicts the X-ray powder diffraction (XRPD) pattern of crystalline form 2-A, according to one set of embodiments.

FIG. 15 depicts the XRPD pattern of crystalline form 3-A, according to one set of embodiments.

FIG. 16 depicts the XRPD pattern of crystalline form 3-B, according to one set of embodiments.

FIG. 17 depicts the XRPD pattern of crystalline form 1-B, according to one set of embodiments.

### DETAILED DESCRIPTION

A pharmaceutical composition described herein (referred to herein as a “subject composition”) includes a drug-containing particle having a modification to a property of its surface. Although there are a number of surface properties that may be modified, some embodiments relate to surfaces that are modified to provide reduced adhesion to mucus or improved penetration of the particles through physiological mucus, as compared to unmodified drug-containing particles. Thus, disclosed herein are subject compositions comprising mucus-penetrating particles comprising a pharmaceutical composition coated with a mucus penetration-enhancing surface-altering agent.

Particles having efficient transport through mucus barriers may be referred to herein as mucus-penetrating particles (MPPs). The particles may more readily penetrate the mucus layer of a tissue to avoid or minimize mucus adhesion and/or rapid mucus clearance. Therefore, drugs contained in MPPs may be more effectively delivered to, and may be retained longer in, the target issue. As a result, the drugs contained in MPPs may be administered at a lower dose and/or less frequently than formulations lacking MMPs to achieve similar or superior exposure. Moreover, the relatively low and/or infrequent dosage of the subject compositions may result in fewer or less severe side effects, and/or improved patient compliance.

Non-limiting examples of mucosal tissues include oral (e.g., including the buccal and esophageal membranes and tonsil surface), ophthalmic, gastrointestinal (e.g., including stomach, small intestine, large intestine, colon, rectum), nasal, respiratory (e.g., including nasal, pharyngeal, tracheal and bronchial membranes), and genital (e.g., including vaginal, cervical and urethral membranes) tissues.

Examples of pharmaceutical applications that may benefit from these properties include including drug delivery, imaging, and diagnostic applications. For example, a subject composition may be well-suited for ophthalmic applications, and may be used for delivering pharmaceutical agents to the front of the eye, middle of the eye, and/or the back of the eye. With respect to the front of the eye, MPPs may reduce dosage frequency because lower adhesion to mucus may allow the drug to be more evenly spread across the surface of the eye, thereby avoiding the eye's natural clearance mechanisms and prolonging their residence at the ocular surface. Improved mucus penetration allows the drug to penetrate through the mucus coating of the eye more quickly. With respect to the back of the eye, MPPs may allow improved delivery so that a therapeutically effective amount of a drug can reach the back of the eye. In some embodiments, MPPs may effectively penetrate through physiological mucus to facilitate sustained drug release directly to the underlying tissues, as described in more detail below. Mucus-penetrating particles are further disclosed in US Patent application publications 2013/0316009, 2013/01316006, and 2015/0125539, and U.S. Pat. No. 9,056,057, incorporated by reference herein for all they disclose regarding mucus-penetrating particles.

### Coated Particles

In some embodiments, the particles described herein have a core-shell type arrangement. The core may comprise any suitable material such as a solid pharmaceutical agent having a relatively low aqueous solubility, a polymeric carrier, a lipid, and/or a protein. The core may also comprise a gel or a liquid in some embodiments. The core may be coated with a coating or shell comprising a mucus penetration-enhancing surface-altering agent that facilitates mobility of the particle in mucus. As described in more detail below, in some embodiments the mucus penetration-enhancing surface-altering agent may comprise a polymer (e.g., a synthetic or a natural polymer) having pendant hydroxyl groups on the backbone of the polymer. The molecular weight and/or degree of hydrolysis of the polymer may be chosen to impart certain transport characteristics to the particles, such as increased transport through mucus. In certain embodiments, the mucus penetration-enhancing surface-altering agent may comprise a triblock copolymer comprising a hydrophilic block-hydrophobic block-hydrophilic block configuration. The molecular weights of each of the blocks may be chosen to impart certain transport characteristics to the particles, such as increased transport through mucus. In certain embodiments, the mucus penetration-enhancing surface-altering agent may comprise a polysorbate.

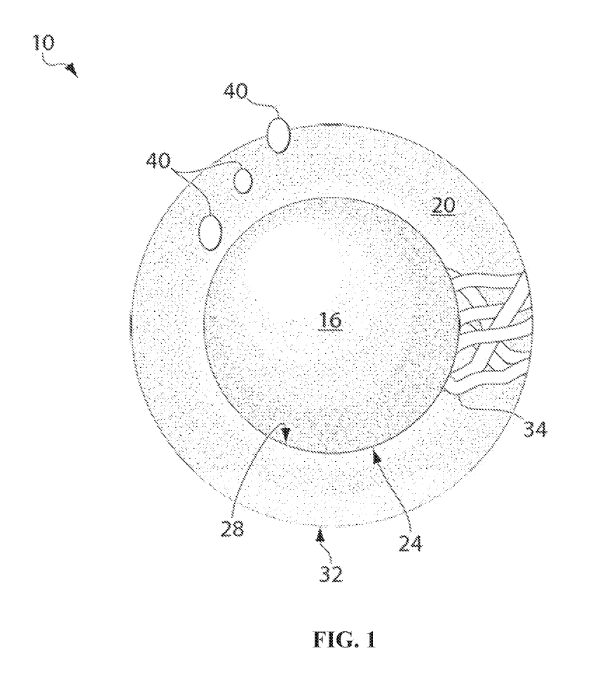

Some embodiments of a coated particle are depicted in FIG. 1. In FIG. 1, particle 10 includes a core 16 (which may be in the form of a particle) and a coating 20 surrounding the core. The core includes a surface 24 to which one or more surface-altering agents can be attached or adhered. For instance, in some cases, core 16 is surrounded by coating 20, which includes an inner surface 28 and an outer surface 32. The coating may comprise one or more surface-altering agents 34, such as a polymer (e.g., a block copolymer and/or a polymer having pendant hydroxyl groups), which may associate with surface 24 of the core. Particle 10 may optionally include one or more components 40 such as targeting moieties, proteins, nucleic acids, and bioactive agents which may optionally impart specificity to the particle. For example, a targeting agent or molecule (e.g., a protein, nucleic acid, nucleic acid analog, carbohydrate, or small molecule), if present, may aid in directing the particle to a specific location in the subject's body. The location may be, for example, a tissue, a particular cell type, or a subcellular compartment. One or more components 40, if present, may be associated with the core, the coating, or both; e.g., they may be associated with surface 24 of the core, inner surface 28 of the coating, outer surface 32 of the coating, and/or embedded in the coating. The one or more components 40 may be associated through covalent bonds, absorption, or attached through ionic interactions, hydrophobic and/or hydrophilic interactions, electrostatic interactions, van der Waals interactions, or combinations thereof. In some embodiments, a component may be attached (e.g., covalently) to one or more of the surface-altering agents of the coated particle.

In certain embodiments, a particle described herein has certain a relative velocity, <Vₘₑₐₙ>ᵣₑₗ, which is defined as follows:

$$\text{<} {V}_{mean} {\text{>}}_{rel} = \frac{\text{<} {V}_{mean} {\text{>}}_{Sample} - \text{<} {V}_{mean} {\text{>}}_{Negative control}}{\text{<} {V}_{mean} {\text{>}}_{Positive control} - \text{<} {V}_{mean} {\text{>}}_{Negative control}} \qquad ( Equation 1 )$$

where <Vₘₑₐₙ> is the ensemble average trajectory-mean velocity, Vₘₑₐₙ is the velocity of an individual particle averaged over its trajectory, the sample is the particle of interest, the negative control is a 200 nm carboxylated polystyrene particle, and the positive control is a 200 nm polystyrene particle densely PEGylated with 2 kDa-5 kDa PEG.

The relative velocity can be measured by a multiple particle tracking technique. For instance, a fluorescent microscope equipped with a CCD camera can be used to capture 15 sec movies at a temporal resolution of 66.7 msec (15 frames/sec) under 100× magnification from several areas within each sample for each type of particles: sample, negative control, and positive control. The sample, negative and positive controls may be fluorescent particles to observe tracking. Alternatively non-fluorescent particles may be coated with a fluorescent molecule, a fluorescently tagged surface agent or a fluorescently tagged polymer. An advanced image processing software (e.g., Image Pro or MetaMorph) can be used to measure individual trajectories of multiple particles over a time-scale of at least 3.335 sec (50 frames).

In some embodiments, a MPP described herein has a relative velocity, or a mean relative velocity, in mucus, of at least about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0; up to: about 10.0, about 8.0, about 6.0, about 4.0, about 3.0, about 2.0, about 1.9, about 1.8, about 1.7, about 1.6, about 1.5, about 1.4, about 1.3, about 1.2, about 1.1, about 1.0, about 0.9, about 0.8, or about 1.7; about 0.5-6, or any velocity in a range bounded by any of these values.

In certain embodiments, an MPP described herein can diffuse through mucus or a mucosal barrier at a greater rate or diffusivity, or may have a greater geometric mean squared displacement, than a control particle or a corresponding particle (e.g., a corresponding particle that is unmodified and/or is not coated with a coating described herein). In some cases, a particle described herein may pass through mucus or a mucosal barrier at a rate of diffusivity, or with a geometric mean squared displacement, that is at least about 10 times, 20 times, 30 times, 50 times, 100 times, 200 times, 500 times, 1000 times, 2000 times, 5000 times, 10000 times, or more; up to about 10000 times, about 5000 times, about 2000 times, about 1000 times, about 500 times, about 200 times, about 100 times, about 50 times, about 30 times, about 20 times, about 10 times; about 10-1000 times higher than a control particle or a corresponding particle; or may have any increase in diffusivity in a range bounded by any of these values.

In some embodiments, an MPP described herein diffuses through a mucosal barrier at a rate approaching the rate or diffusivity at which the particles can diffuse through water. In some cases, a particle described herein may pass through a mucosal barrier at a rate or diffusivity that is at least about 1/10,000, about 1/5000, about 1/2000, about 1/1000, about 1/900, about 1/800, about 1/700, about 1/600, about 1/500, about 1/400, about 1/300, about 1/200, or about 1/100; up to about 1/100, about 1/200, about 1/300, about 1/400, about 1/500, about 1/600, about 1/700, about 1/800, about 1/900, about 1/1000, about 1/2000, about 1/5000, about 1/10; or 1/5000-1/500, the diffusivity that the particle diffuses through water under identical conditions, or any rate or diffusivity in a range bounded by any of these values.

In a particular embodiment, an MPP described herein may diffuse through human mucus at a diffusivity that is less than about 1/500 the diffusivity that the particle diffuses through water. In some cases, the measurement is based on a time scale of about 1 second, or about 0.5 second, or about 2 seconds, or about 5 seconds, or about 10 seconds.

In certain embodiments provided herein particles travel through mucus at certain absolute diffusivities. For example, the MPPs described herein may travel at diffusivities of at least about 1×10⁻⁴ μm/s, 2×10⁻⁴ μm/s, 5×10⁻⁴ μm/s, 1×10⁻³ μm/s, 2×10⁻³ μm/s, 5×10⁻³ μm/s, 1×10⁻² μm/s, 2×10⁻² μm/s, 4×10⁻² μm/s, 5×10⁻² μm/s, 6×10⁻² μm/s, 8×10⁻² μm/s, 1×10⁻¹ μm/s, 2×10⁻¹ μm/s, 5×10⁻¹ μm/s, 1 μm/s, or 2 μm/s; up to about 2 μm/s, about 1 μm/s, about 5×10⁻¹ μm/s, about 2×10⁻¹ μm/s, about 1×10⁻¹ μm/s, about 8×10⁻² μm/s, about 6×10⁻² μm/s, about 5×10⁻² μm/s, about 4×10⁻² μm/s, about 2×10⁻² μm/s, about 1×10⁻² μm/s, about 5×10⁻³ μm/s, about 2×10⁻³ μm/s, about 1×10⁻³ μm/s, about 5×10⁻⁴ μm/s, about 2×10⁻⁴ μm/s, or about 1×10⁻⁴ μm/s; or about 2×10⁻⁴-1×10⁻¹ μm/s, or any absolute diffusivity in a range bounded by any of these values. In some cases, the measurement is based on a time scale of about 1 second, or about 0.5 second, or about 2 seconds, or about 5 seconds, or about 10 seconds.

In some embodiments, a subject composition comprises a plurality of particles coated with a mucus penetration-enhancing coating comprising a surface-altering agent, such as a plurality of coated particles. Such a coated particle contains a core comprising the drug and a coating comprising a surface-altering agent.

The surface-altered particles, such as the coated particles described herein, may have any suitable shape and/or size. In some embodiments, a coated particle has a shape substantially similar to the shape of the core. In some cases, a coated particle described herein may be a nanoparticle, i.e., the particle has a characteristic dimension of less than about 1 micrometer, where the characteristic dimension of the particle is the diameter of a perfect sphere having the same volume as the particle. In other embodiments, larger sizes are possible. A plurality of particles, in some embodiments, may also be characterized by an average size, an average characteristic dimension, an average largest cross-sectional dimension, or an average smallest cross-sectional dimension of less than or equal to about 10 μm, less than or equal to about 5 μm, less than or equal to about 1 μm, about 700-800 nm, about 500-700 nm, about 400-500 nm, about 300-400 nm, about 200-300 nm, about 50-200 nm, about 5-100 nm, about 50-75 nm, about 5-50 nm, about 5-40 nm, about 5-35 nm, about 5-30 nm, about 5-25 nm, about 5-20 nm, about 5-15 nm, about 0.1-5 nm, about 200-400 nm, about 200-500 nm, about 100-400 nm, or about 100-300 nm; at least about 5 nm, at least about 20 nm, at least about 50 nm, about 100-700 nm, about 200-500 nm, about 5 μm, about 10 nm, about 1 μm, about 10 nm-5 μm, 50-500 nm, 200-500 nm, about 1-10 μm or any size in a range bounded by any of these values. In some embodiments, the sizes of the cores formed by a process described herein have a Gaussian-type distribution.

It is appreciated in the art that the ionic strength of a formulation comprising particles may affect the polydispersity of the particles. Polydispersity is a measure of the heterogeneity of sizes of particles in a formulation. Heterogeneity of particle sizes may be due to differences in individual particle sizes and/or to the presence of aggregation in the formulation. A formulation comprising particles is considered substantially homogeneous or “monodisperse” if the particles have essentially the same size, shape, and/or mass. A formulation comprising particles of various sizes, shapes, and/or masses is deemed heterogeneous or “polydisperse”.

In some embodiments, the polydispersity index of a subject composition, such as a polydispersity index of a particle size or a molecular weight, is at least about 0.005, about 0.01, about 0.05, about 0.1, about 0.15, about 0.2, about 0.3, about 0.4, about 0.5, about 0.6, about 0.7, about 0.8, about 0.9, or about 1; up to about 1, about 0.9, about 0.8, about 0.7, about 0.6, about 0.5, about 0.4, about 0.3, about 0.2, about 0.15, about 0.1, about 0.05, about 0.01, or about 0.005; about 0.1-0.5, about 0.1, about 0.15, about 0.2, or any polydispersity index in a range bounded by any of these values. Polydispersity index may be determined according to ISO standards ISO 13321:1996 E and ISO 22412:2008.

Although many methods for determining sizes of particles are known, the sizes described herein (e.g., average particle sizes, thicknesses) refer to ones measured by dynamic light scattering.

The MPPs may result in a subject composition that is capable of sustaining a therapeutically effective level, or delivering a therapeutically effect amount, of the pharmaceutical agent, such as a hydrocortisone derivative, in a target tissue. For example, an ophthalmically effective level or an ophthalmically effective amount of the drug-containing MPP may be delivered to an ocular tissue, e.g. an anterior ocular tissue, such as a palpebral conjunctiva, a bulbar conjunctiva, a fornix conjunctiva, an aqueous humor, an anterior sclera, a cornea, an iris, or a ciliary body; or the back of the eye, such as a vitreous humor, a vitreous chamber, such as a retina, a macula, a choroid, a posterior sclera, a uvea, an optic nerve, or the blood vessels or nerves which vascularize or innervate a posterior ocular region or site. In some embodiments, the concentration of the pharmaceutical agent, such as a hydrocortisone derivative, in the tissue may be increased by at least about 10%, about 20%, about 30%, about 40%, about 50%, about 60% or more, within a short relatively amount of time, compared to the concentration of the pharmaceutical agent when administered without the mucus penetration-enhancing coating.

A subject composition may increase the drug level, e.g. the hydrocortisone derivative level, within a relatively short amount of time, such as within about 24 hours, about 18 hours, about 12 hours, about 9 hours, about 6 hours, about 4 hours, about 3 hours, about 2 hours, about 1 hour, about 30 minutes, about 20 minutes, about 10 minutes, about 10 minutes to about 2 hours, or any time in a range bounded by any of these values.

A subject composition may achieve therapeutically effective level or an ophthalmically effective level of hydrocortisone derivatives, potentially as a result of the mucus penetration-enhancing coating of the MPP, for a sustained period of time after administration, such as least: 10 minutes, 20 minutes, 30 minutes, 1 hour, 2 hours, 4 hours, 6 hours, 9 hours, 12 hours, 18 hours, 1 day, 2 days, 3 days, 4 days, 5 days, 6 days, or 1 week; up to: 1 week, 6 days, 5 days, 4 days, 3 days, 2 days, 1 day, 12 hours, 9 hours, 6 hours, 4 hours, 2 hours, 1 hour; or about 4 hours to about 1 week, about 10 minutes to about 2 hours, or any time in a range bounded by any of these values.

The core may contain particles of pharmaceutical agents that have a low aqueous solubility, such as a hydrocortisone derivative disclosed below and in U.S. Pat. No. 8,906,892 which is incorporated herein by reference for all it discloses regarding hydrocortisone derivatives. The hydrocortisone derivative may be in a crystalline or nanocrystalline (including any polymorph form) or an amorphous form. In some embodiments, the hydrocortisone derivative is (10R,11S,13S,17R)-11-hydroxy-17-(2-hydroxyacetyl)-10,13-dimethyl-3-oxo-2,3,6,7,8,9,10,11,12,13,14,15,16,17-tetradecahydro-1H-cyclopenta[a]phenanthren-17-yl 3-(phenylsulfonyl)propanoate (Compound 1), (10R,11S,13S,17R)-11-hydroxy-17-(2-hydroxyacetyl)-10,13-dimethyl-3-oxo-2,3,6,7,8,9,10,11,12,13,14,15,16,17-tetradecahydro-1H-cyclopenta[a]phenanthren-17-yl furan-2-carboxylate (Compound 2), or (10R,11S,13S,17R)-11-hydroxy-17-(2-hydroxyacetyl)-10,13-dimethyl-3-oxo-2,3,6,7,8,9,10,11,12,13,14,15,16,17-tetradecahydro-1H-cyclopenta[a]phenanthren-17-yl 2-(4-bromophenyl)acetate (Compound 3).

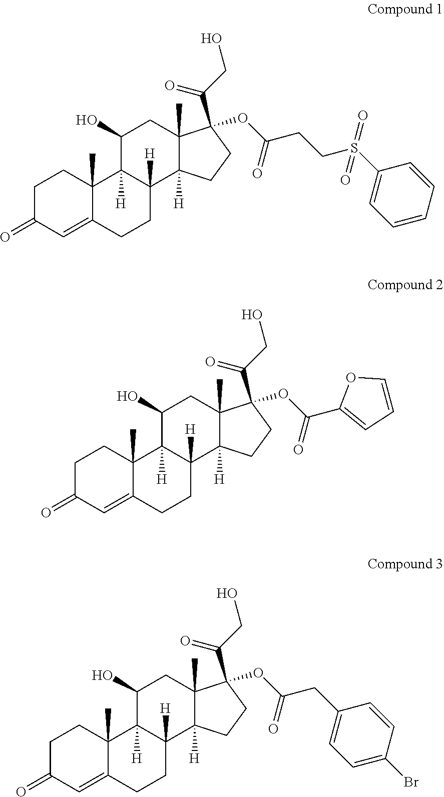

Unless otherwise indicated, any reference to a compound herein, such as a hydrocortisone derivative, by structure, name, or any other means, includes prodrugs, such as ester prodrugs; alternate solid forms, such as polymorphs, solvates, hydrates, etc.; tautomers; or any other chemical species that may rapidly convert to a compound described herein under conditions in which the compounds are used as described herein.

The core may comprise the pharmaceutical agent, such as a hydrocortisone derivative. The core may be substantially all pharmaceutical agent, or may comprise additional components, such as a polymer, a lipid, a protein, a gel, a liquid, a surfactant, a tonicity agent (such as glycerin), a buffer, a salt (such as NaCl), a preservative (such as benzalkonium chloride), a chelating agent (such as EDTA), a filler, etc. In some embodiments, the core particles comprise a hydrocortisone derivative that is encapsulated in a polymer, a lipid, a protein, or a combination thereof. In various embodiments the term encapsulation encompasses any or all of a coating or shell of the encapsulating substance surrounding the rest of the core particle, a solidified co-solution comprising the encapsulating substance and the hydrocortisone derivative of the core particle, a dispersion of the hydrocortisone derivative within a matrix comprising the encapsulating substance, and the like.

In embodiments in which the core particles comprise relatively high amounts of a hydrocortisone derivative disclosed herein (e.g., at least about 50 wt % of the core particle), the core particles generally have an increased loading of a hydrocortisone derivative compared to particles that are formed by encapsulating agents into polymeric carriers. This is an advantage for drug delivery applications, since higher drug loadings mean that fewer numbers of particles may be needed to achieve a desired effect compared to the use of particles containing polymeric carriers.

Suitable polymers for use in a core may include a synthetic polymer, e.g. non-degradable polymers such as polymethacrylate and degradable polymers such as polylactic acid, polyethylene glycol, polyglycolic acid and copolymers thereof (such as PLA-PEG), and/or a natural polymer, such as hyaluronic acid, chitosan, and collagen, or a mixture of polymers.

A core may comprise a biodegradable polymer such as poly(ethylene glycol)-poly(propylene oxide)-poly(ethylene glycol) triblock copolymers, poly(lactide) (or poly(lactic acid)), poly(glycolide) (or poly(glycolic acid)), poly(orthoesters), poly(caprolactones), polylysine, poly(ethylene imine), poly(acrylic acid), poly(urethanes), poly(anhydrides), poly(esters), poly(trimethylene carbonate), poly(ethyleneimine), poly(acrylic acid), poly(urethane), poly(beta amino esters) or the like, and combinations, copolymers or derivatives of these and/or other polymers, for example, poly(lactide-co-glycolide) (PLGA).

In certain embodiments, a polymer may biodegrade within a period that is acceptable in the desired application. In certain embodiments, such as in vivo therapy, such degradation occurs in a period usually less than about five years, one year, six months, three months, one month, fifteen days, five days, three days, or even one day or less (e.g., 1-4 hours, 4-8 hours, 4-24 hours, 1-24 hours) on exposure to a physiological solution with a pH between 6 and 8 having a temperature of between 25 and 37° C. In some embodiments, the polymer degrades in a period of between about one hour and several weeks.

The pharmaceutical agent may be present in the core in any suitable amount, e.g., at about 1-100 wt %, 5-100 wt %, 10-100 wt %, 20-100 wt %, 30-100 wt %, 40-100 wt %, 50-100 wt %, 60-100 wt %, 70-100 wt %, 80-100 wt %, 85-100 wt %, 90-100 wt %, 95-100 wt %, 99-100 wt %, 50-90 wt %, 60-90 wt %, 70-90 wt %, 80-90 wt %, 85-90 wt % of the core, 70 wt %, 75 wt %, 80 wt %, 85 wt %, 90 wt %, 95 wt %, 97 wt %, or any amount in a range bounded by any of these values.

If a polymer is present in the core, the polymer may be present in the core in any suitable amount, e.g., 1-20%, 20-40%, 40-60%, 60-80%, or 80-95% by weight, or any amount in a range bounded by any of those values. In one set of embodiments, the core is formed is substantially free of a polymeric component.

The core may have any suitable shape and/or size. For instance, the core may be substantially spherical, non-spherical, oval, rod-shaped, pyramidal, cube-like, disk-shaped, wire-like, or irregularly shaped. The core may have a largest or smallest cross-sectional dimension of, for example, less than or equal to: about 10 μm, about 5 μm, about 1 μm, about 5-800 nm, about 5-700 nm, about 5-500 nm, about 400 nm, or about 300 nm; 5-200 nm, 5-100 nm, 5-75 nm, 5-50 nm, 5-40 nm, 5-35 nm, 5-30 nm, 5-25 nm, 5-20 nm, 5-15 nm, about 50-500 nm, at least: about 20 nm, about 50 nm, about 100 nm, about 200 nm, about 300 nm, about 400 nm, at least about 500 nm, about 1 μm, or about 5 μm, or any size in a range bounded by any of these values. In some embodiments, the sizes of the cores formed by a process described herein have a Gaussian-type distribution.

The surface of a core may be partially or completely covered by a mucus penetration-enhancing coating. The coating may comprise a surface-altering agent, which may be any agent that modifies the surface of the core particles to reduce the adhesion of the particles to mucus and/or to facilitate penetration of the particles through physiological mucus.

In some embodiments, hydrophobic portions of a mucus penetration-enhancing surface-altering agent (e.g., non-hydrolyzed portions of polyvinyl alcohol, hydrophobic polyalkylene oxide, etc.) may allow the polymer to be adhered to the core surface (e.g., in the case of the core surface being hydrophobic), thus allowing for a strong association between the core and the polymer.

In some embodiments, hydrophilic portions of a surface-altering agent (e.g. hydrolyzed portions of polyvinyl alcohol, polethylene oxide, etc.) can render the surface-altering agent, and as a result the particle, hydrophilic. The hydrophilicity may shield the coated particles from adhesive interactions with mucus, which may help to improve mucus transport or penetration.

Examples of suitable surface-altering agents include a block copolymer having one or more relatively hydrophilic blocks and one or more relatively hydrophobic blocks, such as a triblock copolymer, wherein the triblock copolymer comprises a hydrophilic block-hydrophobic block-hydrophilic block configuration, a diblock copolymer having a hydrophilic block-hydrophobic block configuration; a combination of a block copolymer with one or more other polymers suitable for use in a coating; a polymer-like molecule having a nonlinear block configurations, such as nonlinear configurations of combinations of hydrophilic and hydrophobic blocs, such as a comb, a brush, or a star copolymer; a synthetic polymer having pendant hydroxyl groups on the backbone of the polymer; a polysorbate; a surfactant; etc.

The surface-altering agent may have any suitable molecular weight, such as at least about 1 kDa, about 2 kDa, about 4 kDa, about 5 kDa, about 8 kDa, about 9 kDa, about 10 kDa, about 12 kDa, about 15 kDa about 20 kDa, about 25 kDa, about 30 kDa, about 40 kDa, about 50 kDa, about 60 kDa, about 70 kDa, about 80 kDa, about 90 kDa, about 100 kDa about 110 kDa, about 120 kDa, about 130 kDa, about 140 kDa, about 150 kDa, about 200 kDa, about 500 kDa, or about 1000 kDa; less than or equal to about 1000 kDa, about 500 kDa, about 200 kDa, about 180 kDa, about 150 kDa, about 130 kDa, about 120 kDa, about 100 kDa, about 85 kDa, about 70 kDa, about 65 kDa, about 60 kDa, about 50 kDa, about 40 kDa, about 30 kDa, about 20 kDa, about 15 kDa, about 10 kDa; about 10-30 kDa, about 1-100 kDa, about 1-50 kDa, about 1-3 kDa, about 2-7 kDa, about 5-10 kDa, about 8-12 kDa, about 9-15 kDa, about 10-15 kDa, about 12-17 kDa, about 15-25 kDa about 20-30 kDa, about 25-40 kDa, about 30-50 kDa, about 40-60 kDa, about 50-70 kDa; or a molecular weight in a range bounded by any of these values.

When the surface-altering agent is a block copolymer, the molecular weight of the hydrophilic blocks and the hydrophobic blocks of the block copolymers, or the relative amount of the hydrophobic block with respect to the hydrophilic block, may affect the mucoadhesion and/or mucus penetration of a core and association of the block copolymer with the core. Many block copolymers comprise a polyether portion, such as a polyalkylether portion. A polyether block may be relatively hydrophilic (e.g. polyethylene glycol) or relatively hydrophobic (e.g. polyalkylene glycols based upon monomer or repeating units having 3 or more carbon atoms).

The copolymer may have any suitable molecular weight, such as at least about 1 kDa, about 2 kDa, about 4 kDa, about 5 kDa, about 8 kDa, about 9 kDa, about 10 kDa, about 12 kDa, about 15 kDa about 20 kDa, about 25 kDa, about 30 kDa, about 40 kDa, about 50 kDa, about 60 kDa, about 70 kDa, about 80 kDa, about 90 kDa, about 100 kDa about 110 kDa, about 120 kDa, about 130 kDa, about 140 kDa, about 150 kDa, about 200 kDa, about 500 kDa, or about 1000 kDa; less than or equal to about 1000 kDa, about 500 kDa, about 200 kDa, about 180 kDa, about 150 kDa, about 130 kDa, about 120 kDa, about 100 kDa, about 85 kDa, about 70 kDa, about 65 kDa, about 60 kDa, about 50 kDa, about 40 kDa, about 30 kDa, about 20 kDa, about 15 kDa, about 10 kDa; about 10-30 kDa, about 1-100 kDa, about 1-50 kDa, about 1-3 kDa, about 2-7 kDa, about 5-10 kDa, about 8-12 kDa, about 9-15 kDa, about 10-15 kDa, about 12-17 kDa, about 15-25 kDa about 20-30 kDa, about 25-40 kDa, about 30-50 kDa, about 40-60 kDa, about 50-70 kDa; or a molecular weight in a range bounded by any of these values.

A hydrophobic block may be any suitable block in a block copolymer that is relatively hydrophobic as compared to another block in the copolymer. The hydrophobic block may be substantially present in the interior of the coating and/or at the surface of the core particle, e.g., to facilitate attachment of the coating to the core. Examples of suitable polymers for use in the hydrophobic block include polyalkylethers having 3 or more carbon atoms in each repeating unit, such as polypropylene glycol, polybutylene glycol, polypentylene glycol, polyhexylene glycol, etc.; esters of polyvinyl alcohol such as polyvinyl acetate; polyvinyl alcohol having a low degree of hydrolysis, etc.

Any suitable amount of the hydrophobic blocks may be used. For example, the hydrophobic block may be a sufficiently large portion of the polymer to allow the polymer to adhere to the core surface, particularly if the core surface is hydrophobic. In certain embodiments, the molecular weight of the (one or more) relatively hydrophobic blocks of a block copolymer, such as poly(propylene oxide) (PPO), is at least about 0.5 kDa, about 1 kDa, about 2 kDa, about 3 kDa, about 4 kDa, about 5 kDa, about 6 kDa, about 10 kDa, about 12 kDa, about 15 kDa, about 20 kDa, about 50 kDa, about 60 kDa, about 70 kDa, about 80 kDa, about 90 kDa, about 100 kDa about 110 kDa, about 120 kDa, about 130 kDa, about 140 kDa, about 150 kDa, about 200 kDa, about 500 kDa, about 1000 kDa; up to about 1000 kDa, about 500 kDa, about 200 kDa, about 150 kDa, about 140 kDa, about 130 kDa, about 120 kDa, about 110 kDa, about 100 kDa, about 90 kDa, about 80 kDa, about 50 kDa, about 20 kDa, about 15 kDa, about 13 kDa, about 12 kDa, about 10 kDa, about 8 kDa, or about 6 kDa; or about 3-15 kDa, 0.5-5 kDa, 0.5-1 kDa, 1-2 kDa, 2-3 kDa, 2-2.5 kDa, 2.5-3 kDa, 3-8 kDa, 3-3.5 kDa, 3.5-4 kDa, 3-4 kDa, 4-5 kDa, about 0.5-3 kDa, 2.5-3 kDa, 2.7-3 kDa, 2.8-3 kDa, 3-3.3 kDa, 3-3.5 kDa, 3.5-3.7 kDa, 3.5-4 kDa, 5-4.5 kDa, 5-10 kDa, or any molecular weight in a range bounded by any of these values.

A hydrophilic block may be any suitable block in a block copolymer that is relatively hydrophilic as compared to another block in the block copolymer. In some cases, the hydrophilic blocks may be substantially present at the outer surface of the particle. For example, the hydrophilic blocks may form a majority of the outer surface of the coating and may help stabilize the particle in an aqueous solution containing the particle. Examples of suitable polymers for use in the hydrophilic block include polyethylene glycol, or synthetic polymers having hydroxyl pendant groups such as polyvinyl alcohol having a high degree of hydrolysis. Any suitable amount of the hydrophilic block may be used, such as an amount that is sufficiently large to render the coated particle hydrophilic when present at the surface of the particle.

In some embodiments, the combined (one or more) relatively hydrophilic blocks, e.g. PEO or polyvinyl alcohol, or repeat units of a block copolymer constitute at least about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, or about 70 wt %; up to about 90 wt %, about 80 wt %, about 60 wt %, about 50 wt %, or about 40 wt % of the block copolymer; or about 30-80 wt %, about 10-30 wt %, 10-40 wt %, about 30-50 wt %, about 40-80 wt %, about 50-70 wt %, about 70-90 wt %, about 15-80 wt %, about 20-80 wt %, about 25-80 wt %, about 30-80 wt %, of the block copolymer, or any percentage in a range bounded by any of these values.

In some embodiments, the molecular weight of the (one or more) relatively hydrophilic blocks or repeat units, such as poly(ethylene oxide) (PEO) or poly(vinyl alcohol) (PVA), of the block copolymer may be at least about 0.5 kDa, about 1 kDa, about 2 kDa, about 3 kDa, about 4 kDa, about 5 kDa, about 6 kDa, about 10 kDa, about 12 kDa, about 15 kDa, about 20 kDa, or about 50 kDa, about 60 kDa, about 70 kDa, about 80 kDa, about 90 kDa, about 100 kDa about 110 kDa, about 120 kDa, about 130 kDa, about 140 kDa, about 150 kDa, about 200 kDa, about 500 kDa, or about 1000 kDa; up to about 1000 kDa, about 500 kDa, about 200 kDa, about 150 kDa, about 140 kDa, about 130 kDa, about 120 kDa, about 110 kDa, about 100 kDa, about 90 kDa, about 80 kDa, about 50 kDa, about 20 kDa, about 15 kDa, about 13 kDa, about 12 kDa, about 10 kDa, about 8 kDa, about 6 kDa, about 5 kDa, about 3 kDa, about 2 kDa, about 1 kDa; about 1-2 kDa, about 2-4 kDa, about 3-15 kDa, about 4-7 kDa, 7-10 kDa, about 10-12 kDa, about 10-15 kDa, or any molecular weight in a range bounded by any of these values.

In embodiments in which two hydrophilic blocks flank a hydrophobic block, the molecular weights, and the chemical identity, of the two hydrophilic blocks may be substantially the same or different.

In certain embodiments, the polymer is a triblock copolymer of a polyalkyl ether (e.g., polyethylene glycol, polypropylene glycol) and another polymer (e.g., a synthetic polymer having pendant hydroxyl groups on the backbone of the polymer (e.g., PVA). In certain embodiments, the polymer is a triblock copolymer of a polyalkyl ether (such as polyethylene glycol) and another polyalkyl ether. In certain embodiments, the polymer includes a polypropylene glycol unit flanked by two more hydrophilic units. In certain embodiments, the polymer includes two polyethylene glycol units flanking a more hydrophobic unit. The molecular weights of the two blocks flanking the central block may be substantially the same or different.

In certain embodiments, the polymer is of Formula 1:

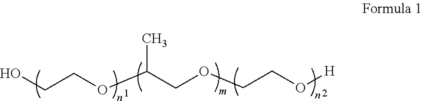

With respect to Formula 1, m is 2-1730, 5-70, 5-100, 20-100, 10-20, 20-30, 30-40, 40-50, 50-60, 60-70, 10-50, 40-60, 50-70, 50-100, 100-300, 300-500, 500-700, 700-1000, 1000-1300, 1300-1600, 1600-2000, about 15, about 20, about 31, about 41, about 51, about 61, about 68, or any integer in a range bounded by any of these values.

With respect to Formula 1, n¹ and n² may be the same or different. In some embodiments, n¹+n², is 2-1140, 2-10, 10-30, 30-40, 40-70, 70-150, 150-200, 10-170, 50-150, 90-110, 100-200, 200-400, 400-600, 600-800, 800-1000, 1000-1500, about 2, about 6, about 8, about 9, about 18, about 29, about 35, about 39, about 41, about 68, about 82, about 127, about 164, about 191, or any integer in a range bounded by any of these values. In certain embodiments, n¹+n² is at least 2 times m, 3 times m, or 4 times m.

With respect to Formula 1, in some embodiments m is about 10-30 and n¹+n² is about 2-10, m is about 10-30 and n¹+n² is about 10-30, m is about 30-50 and n¹+n² is about 2-10, m is about 40-60 and n¹+n² is about 2-10, m is about 30-50 and n¹+n² is about 40-100, m is about 60-80 and n¹+n² is about 2-10, m is about 40-60 and n¹+n² is about 20-40, m is about 10-30 and n¹+n² is about 10-30, m is about 60-80 and n¹+n² is about 20-40, m is about 40-60 and n¹+n² is about 40-100, m is about 30-50 and n¹+n² is about 100-200, m is about 30-50 and n¹+n² is about 100-200, m is about 60-80 and n¹+n² is about 100-200, or m is about 60-80 and n¹+n² is about 20-40.

In certain embodiments, the coating includes a surface-altering agent comprising a (poly(ethylene glycol))-(poly(propylene oxide))-(poly(ethylene glycol)) triblock copolymer (hereinafter “PEG-PPO-PEG triblock copolymer”), present in the coating alone or in combination with another polymer such as a synthetic polymer having pendant hydroxyl groups on the backbone of the polymer (e.g., PVA). As described herein, the PEG blocks may be interchanged with PEO blocks in some embodiments. The molecular weights of the PEG (or PEO) and PPO segments of the PEG-PPO-PEG triblock copolymer may be selected so as to reduce the mucoadhesion of the particle, as described herein. Without wishing to be bound by theory, a particle having a coating comprising a PEG-PPO-PEG triblock copolymer may have reduced mucoadhesion as compared to a control particle due to, at least in part, the display of a plurality of PEG (or PEO) segments on the particle surface. The PPO segment may be adhered to the core surface (e.g., in the case of the core surface being hydrophobic), thus allowing for a strong association between the core and the triblock copolymer. In some cases, the PEG-PPO-PEG triblock copolymer is associated with the core through non-covalent interactions. For purposes of comparison, the control particle may be, for example, a carboxylate-modified polystyrene particle of similar size as the coated particle in question.

In some embodiments, a triblock copolymer, such as a PEO-PPO-PEO copolymer, has an average molecular weight that is at least about 1 kDa, about 2 kDa, about 4 kDa, about 5 kDa, about 8 kDa, about 9 kDa, about 10 kDa; less than or equal to about 100 kDa, about 50 kDa, about 20 kDa, about 15 kDa, about 10 kDa; or is about 1-3 kDa, 1-3 kDa, 2-4 kDa, 3-5 kDa, 4-6 kDa, 5-7 kDa, 6-8 kDa, 7-9 kDa, 8-10 kDa, 5-7 kDa, about 2-7 kDa, about 5-10 kDa, about 8-12 kDa, about 9-15 kDa, about 10-15 kDa, about 12-17 kDa, about 15-25 kDa about 20-30 kDa, about 25-40 kDa, about 30-50 kDa, about 40-60 kDa, about 50-70 kDa; or a molecular weight in a range bounded by any of these values.

In certain embodiments, a surface-altering agent includes a polymer comprising a poloxamer, having the trade name Pluronic®. Pluronic® polymers that may be useful in the embodiments described herein include, but are not limited to, F127, F38, F108, F68, F77, F87, F88, F98, F123, L101, L121, L31, L35, L43, L44, L61, L62, L64, L81, L92, N3, P103, P104, P105, P123, P65, P84, and P85.

In some embodiments, the surface-altering agent comprises Pluronic F127, F108, P123, P105, or P103.

Examples of molecular weights of certain Pluronic® molecules are shown in Table 1.

TABLE 1
Molecular Weights of Pluronic ® molecules

| Pluronic ® | Poloxamer | Average MW | MW PPO | PEO wt % | MW PEO |
|---|---|---|---|---|---|
| L31 | 101 | 1000 | 900 | 10 | 100 |
| L44 | 124 | 2000 | 1200 | 40 | 800 |
| L81 | 231 | 2667 | 2400 | 10 | 267 |
| L101 | 331 | 3333 | 3000 | 10 | 333 |
| P65 | 185 | 3600 | 1800 | 50 | 1800 |
| L121 | 401 | 4000 | 3600 | 10 | 400 |
| P103 | 333 | 4286 | 3000 | 30 | 1286 |
| F38 | 108 | 4500 | 900 | 80 | 3600 |
| P105 | 335 | 6000 | 3000 | 50 | 3000 |
| F87 | 237 | 8000 | 2400 | 70 | 5600 |
| F68 | 188 | 9000 | 1800 | 80 | 7200 |
| F127 | 407 | 12000 | 3600 | 70 | 8400 |
| P123 | 403 | 5750 | 4030 | 30 | 1730 |
| F108 | 338 | 14600 | 3250 | 80 | 11350 |

A surface-altering agent may include a synthetic polymer having pendant hydroxyl groups on the backbone of the polymer, such as a poly(vinyl alcohol), a partially hydrolyzed poly(vinyl acetate), a copolymer of vinyl alcohol and vinyl acetate, a poly(ethylene glycol)-poly(vinyl acetate)-poly(vinyl alcohol) copolymer, a poly(ethylene glycol)-poly(vinyl alcohol) copolymer, a poly(propylene oxide)-poly(vinyl alcohol) copolymer, a poly(vinyl alcohol)-poly(acryl amide) copolymer, etc.

The synthetic polymer described herein (e.g., one having pendant hydroxyl groups on the backbone of the polymer) may have any suitable molecular weight, such as at least about 1 kDa, about 2 kDa, about 5 kDa, about 8 kDa, about 9 kDa, about 10 kDa, about 12 kDa, about 15 kDa about 20 kDa, about 25 kDa, about 30 kDa, about 40 kDa, about 50 kDa, about 60 kDa, about 70 kDa, about 80 kDa, about 90 kDa, about 100 kDa, about 110 kDa, about 120 kDa, about 130 kDa, about 140 kDa, about 150 kDa, about 200 kDa, about 500 kDa, or about 1000 kDa; up to about 1000 kDa, about 500 kDa, about 200 kDa, about 180 kDa, about 150 kDa, about 130 kDa, about 120 kDa, about 100 kDa, about 85 kDa, about 70 kDa, about 65 kDa, about 60 kDa, about 50 kDa, about 40 kDa, about 30 kDa, about 20 kDa, about 15 kDa, or about 10 kDa; about 1-1000 kDa, about 1-10 kDa, about 5-20 kDa, about 10-30 kDa, about 20-40 kDa, about 30-50 kDa, about 40-60 kDa, about 50-70 kDa, about 60-80 kDa, about 70-90 kDa, about 80-100 kDa, about 90-110 kDa, about 100-120 kDa, about 110-130 kDa, about 120-140 kDa, about 130-150 kDa, about 140-160 kDa, about 150-170 kDa, or any molecular weight in a range bounded by any of these values.

Poly(vinyl alcohol) may be prepared by polymerizing a vinyl ester to produce a poly(vinyl ester), such as poly(vinyl acetate), and then hydrolyzing the ester to leave free pendant hydroxy groups. Partially hydrolyzed PVA comprises two types of repeating units: vinyl alcohol units (which are relatively hydrophilic) and residual vinyl acetate units (which are relatively hydrophobic). Some embodiments may include one or more blocks of vinyl alcohol units and one or more blocks of vinyl acetate units. In certain embodiments, the repeat units form a copolymer, e.g., a diblock, triblock, alternating, or random copolymer.

The amount of hydrolysis, or the percentage of vinyl alcohol units as compared to the total number of vinyl alcohol+vinyl acetate units, may affect or determine the relative hydrophilicity or hydrophobicity of a poly(vinyl alcohol), and can affect the mucus penetration of the particles. It may be helpful for the degree of hydrolysis to be low enough to allow sufficient adhesion between the PVA and the core (e.g., in the case of the core being hydrophobic). It may also be helpful for the degree of hydrolysis to be high enough to enhance particle transport in mucus. The appropriate level of hydrolysis may depend on additional factors such as the molecular weight of the polymer, the composition of the core, the hydrophobicity of the core, etc.

Less than 95% hydrolysis in a poly(vinyl alcohol) may render a particle mucus penetrating. In some embodiments, a synthetic polymer (e.g., PVA or partially hydrolyzed poly(vinyl acetate) or a copolymer of vinyl alcohol and vinyl acetate) may be at least: about 30%, about 35%, about 40%, about 45%, about 50%, about 55%, about 60%, about 65%, about 70%, about 75%, about 80%, about 85%, about 87%, about 90%, about 95%, or about 98% hydrolyzed; up to about 100%, about 98%, about 97%, about 96%, about 95%, about 94%, about 93%, about 92%, about 91%, about 90%, about 87%, about 85%, about 80%, about 75%, about 70%, or about 60% hydrolyzed; about 80-95%, about 30-95%, about 70-94%, about 30-95%, or about 70-94% hydrolyzed, or any percentage in a range bounded by any of these values.

In some embodiments, a synthetic polymer described herein is, or comprises, PVA. PVA is a non-ionic polymer with surface active properties. In some embodiments, the hydrophilic units of a synthetic polymer described herein may be substantially present at the outer surface of the particle.

The molar fraction of the relatively hydrophilic units and the relatively hydrophobic units of a synthetic polymer may be selected so as to reduce the mucoadhesion of a core and to ensure sufficient association of the polymer with the core, respectively. The molar fraction of the relatively hydrophilic units to the relatively hydrophobic units of a synthetic polymer may be, for example, 0.5:1 (hydrophilic units:hydrophobic units), 1:1, 2:1, 3:1, 5:1, 7:1, 10:1, 15:1, 20:1, 25:1, 30:1, 40:1, 50:1, 75:1, 100:1; up to 100:1, 75:1, 50:1, 40:1, 30:1, 25:1, 20:1, 15:1, 10:1, 7:1, 5:1, 3:1, 2:1, or 1:1; 2:1-4:1, 3:1-5:1, 4:1-6:1, 5:1-7:1, 6:1-8-1, 7:1-9:1, 8:1-10:1, 9:1-11:1, 10:1-20:1, 15:1-50:1, 20:1-1000:1, or any molar ratio in a range bounded by any of these values.

Examples of PVA polymers having various molecular weights and degree of hydrolysis are shown in Table 2. The molecular weight (MW) and hydrolysis degree values were provided by the manufacturers.

TABLE 2
Exemplary PVAs.

| PVA acronym* | MW, kDa | Hydrolysis degree, % |
|---|---|---|
| 2K75 | 2 | 75-79 |
| 9K80 | 9-10 | 80 |
| 13K87 | 13-23 | 87-89 |
| 13K98 | 13-23 | 98 |
| 31K87 | 31-50 | 87-89 |
| 31K98 | 31-50 | 98-99 |
| 57K86 | 57-60 | 86-89 |
| 85K87 | 85-124 | 87-89 |
| 85K99 | 85-124 | 99+ |
| 95K95 | 95 | 95 |
| 105K80 | 104 | 80 |
| 130K87 | 130 | 87-89 |

*PVA acronym: XXKYY, where XX stands for the PVA's lower-end molecular weight in kDa and YY stands for the PVA's lower-end hydrolysis in %.

In certain embodiments, the synthetic polymer is represented by Formula 2:

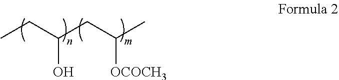

With respect to Formula 2 above, m is 0-11630. Similarly, the value of m may vary. For instance, in certain embodiments, m is at least 5, 10, 20, 30, 50, 70, 100, 150, 200, 250, 300, 350, 400, 500, 800, 1000, 1200, 1500, 1800, 2000, 2200, 2400, 2600, 3000, 5000, 10000, or 15000; up to 15000, 10000, 5000, 3000, 2800, 2400, 2000, 1800, 1500, 1200, 1000, 800, 500, 400, 350, 300, 250, 200, 150, 100, 70, 50, 30, 20, or 10; 5-200, 10-100, 100-150, 150-200, 200-300, 300-400, 400-600, 600-800, 800-1000, 1000-1200, 1200-1400, about 20, about 92, about 102, about 140, about 148, about 247, about 262, about 333, about 354, about 538, about 570, about 611, about 643, about 914, about 972, about 1061, about 1064, about 1333, about 1398, about 1418, or any integer in a range bounded by any of these values.

With respect to Formula 2 above, n is 0-22730. In some embodiments, n is at least 5, 10, 20, 30, 50, 100, 200, 300, 500, 800, 1000, 1200, 1500, 1800, 2000, 2200, 2400, 2600, 3000, 5000, 10000, 15000, 20000, or 25000; up to 30000, 25000, 20000, 25000, 20000, 15000, 10000, 5000, 3000, 2800, 2400, 2000, 1800, 1500, 1200, 1000, 800, 500, 300, 200, 100, or 50; 25-20600, 50-2000, 5-1100, 0-400, 1-400; or 1-10, 10-20, 20-30, 30-50, 50-80, 80-100, 100-150, 150-200, 200-300, about 3, about 5, about 6, about 9, about 10, about 14, about 19, about 23, about 26, about 34, about 45, about 56, about 73, about 87, about 92, about 125, about 182, about 191, about 265, or any integer in a range bounded by any of these values.

It is noted that n and m may represent the total content of the vinyl alcohol and vinyl acetate repeat units in the polymer, or may represent block lengths.

With respect to Formula 2, above, in some embodiments m is about 1-100 and n is about 1-10, m is about 1-100 and n is about 20-30, m is about 100-200 and n is about 20-30, m is about 100-200 and n is about 10-20, m is about 200-300 and n is about 30-50, m is about 100-200 and n is about 1-10, m is about 200-300 and n is about 1-10, m is about 300-500 and n is about 30-50, m is about 500-700 and n is about 70-90, m is about 300-500 and n is about 1-10, m is about 500-700 and n is about 1-10, m is about 500-700 and n is about 70-90, m is about 500-700 and n is about 90-150, m is about 700-100 and n is about 90-150, m is about 1000-1200 and n is about 150-200, m is about 700-100 and n is about 1-10, m is about 1200-1500 and n is about 10-20, m is about 1000-1200 and n is about 50-70, m is about 1000-1200 and n is about 200-300, or m is about 1200-1500 and n is about 150-200.

In some embodiments, the PVA is PVA2K75, PVA9K80, PVA13K87, PVA31K87, PVA57K86, PVA85K87, PVA105K80, or PVA130K87. The PVA acronyms are described using the formula PVAXXKYY, where XX stands for the PVA's lower-end molecular weight in kDa and YY stands for the PVA's lower-end hydrolysis in %.

A surface-altering agent may include a polysorbate. Examples of polysorbates include polyoxyethylene sorbitan monooleate (e.g., Tween® 80), polyoxyethylene sorbitan monostearate (e.g., Tween® 60), polyoxyethylene sorbitan monopalmitate (e.g., Tween® 40), and polyoxyethylene sorbitan monolaurate (e.g., Tween® 20).

In some embodiments, the surface-altering agent comprises a poloxamer, a poly(vinyl alcohol), a polysorbate, or a combination thereof.

In some embodiments, the surface-altering agent comprises L-α-phosphatidylcholine (PC), 1,2-dipalmitoylphosphatidycholine (DPPC), oleic acid, sorbitan trioleate, sorbitan mono-oleate, sorbitan monolaurate, a polyoxylene sorbitan fatty acid ester (Tweens), a polysorbate (e.g., polyoxyethylene sorbitan monooleate) (e.g., Tween® 80), polyoxyethylene sorbitan monostearate (e.g., Tween® 60), polyoxyethylene sorbitan monopalmitate (e.g., Tween® 40), polyoxyethylene sorbitan monolaurate (e.g., Tween® 20), natural lecithin, oleyl polyoxyethylene ether, stearyl polyoxyethylene ether, lauryl polyoxyethylene ether, polyoxylene alkyl ethers, a block copolymer of oxyethylene and oxypropylene, apolyoxyethylene stearate, polyoxyethylene castor oil and/or a derivative thereof, a Vitamin-E-PEG or a derivative thereof, synthetic lecithin, diethylene glycol dioleate, tetrahydrofurfuryl oleate, ethyl oleate, isopropyl myristate, glyceryl monooleate, glyceryl monostearate, glyceryl monoricinoleate, cetyl alcohol, stearyl alcohol, polyethylene glycol, cetyl pyridinium chloride, benzalkonium chloride, olive oil, glyceryl monolaurate, corn oil, cotton seed oil, sunflower seed oil, or a derivative and/or combination thereof.

The surface-altering agent may be present in the pharmaceutical composition in any suitable amount, such as an amount between about 0.001-5%, about 0.001-1%, about 1-2%, about 2-3%, about 3-4%, or about 4-5% by weight.

The surface-altering agent may be present in any suitable amount with respect to the pharmaceutical agent. In some embodiments, the ratio of surface-altering agent to pharmaceutical agent may be at least about 0.001:1 (weight ratio, molar ratio, or w:v ratio), about 0.01:1, about 0.01:1, about 1:1, about 2:1, about 3:1, about 5:1, about 10:1, about 25:1, about 50:1, about 100:1, or about 500:1. In some embodiments, the ratio of surface-altering agent to pharmaceutical agent) is up to about 1000:1 (weight ratio, molar ratio, or w:v ratio), about 500:1, about 100:1, about 75:1, about 50:1, about 25:1, about 10:1, about 5:1, about 3:1, about 2:1, about 1:1, about 0.1:1; and/or about 5:1-50:1, or any ratio in a range bounded by any of these values.

Typically, a coating may be on the surface of, or partially or completely surround or coat, the core. In some embodiments, the surface-altering agent may surround the core particle.

The coating may adhere, or be covalently or non-covalently bound or otherwise attached, to the core. For example, the surface-altering agent may be covalently attached to a core particle, non-covalently attached to a core particle, adsorbed to a core, or coupled or attached to the core through ionic interactions, hydrophobic and/or hydrophilic interactions, electrostatic interactions, van der Waals interactions, or combinations thereof. A surface-altering agent may be oriented in a particular configuration in the coating of the particle. For example, in some embodiments in which a surface-altering agent is a triblock copolymer, such as a triblock copolymer having a hydrophilic block-hydrophobic block-hydrophilic block configuration, and the hydrophobic block may be oriented towards the surface of the core, and the hydrophilic blocks may be oriented away from the core surface (e.g., towards the exterior of the particle).

The coating may include one layer of material (e.g., a monolayer), or multilayers of materials. A single type of surface-altering agent may be present, or multiple types of surface-altering agent.

The surface-altering agent may be present on the surfaces of the core particles at any density that is effective to reduce adhesion to mucus or improved penetration of the particles through mucus. For example, the surface-altering agent may be present on the surfaces of the core particles at a density of at least: about 0.001, about 0.002, about 0.005, about 0.01, about 0.02, about 0.05, about 0.1, about 0.2, about 0.5, about 1, about 2, about 5, about 10, about 20, about 50, or about 100; up to: about 100, about 50, about 20, about 10, about 5, about 2, about 1, about 0.5, about 0.2, about 0.1, about 0.05, about 0.02, or about 0.01; or about 0.01-1 units or molecules/nm²; or any density in a range bounded by any of these values.

Those of ordinary skill in the art will be aware of methods to estimate the average density of surface-altering moieties on the core particle (see, for example, S. J. Budijono et al., Colloids and Surfaces A: Physicochem. Eng. Aspects 360 (2010) 105-110 and Joshi, et al., Anal. Chim. Acta 104 (1979) 153-160, each of which is incorporated herein by reference). For example, as described herein, the average density of surface-altering moieties can be determined using HPLC quantitation and DLS analysis. A suspension of particles for which surface density determination is of interest is first sized using DLS: a small volume is diluted to an appropriate concentration (˜100 μg/mL, for example), and the z-average diameter is taken as a representative measurement of particle size. The remaining suspension is then divided into two aliquots. Using HPLC, the first aliquot is assayed for the total concentration of core material and for the total concentration of surface-altering moiety. Again using HPLC, the second aliquot is assayed for the concentration of free or unbound surface-altering moiety. In order to get only the free or unbound surface-altering moiety from the second aliquot, the particles, and therefore any bound surface-altering moiety, are removed by ultracentrifugation. By subtracting the concentration of the unbound surface-altering moiety from the total concentration of surface-altering moiety, the concentration of bound surface-altering moiety can be determined. Since the total concentration of core material was also determined from the first aliquot, the mass ratio between the core material and the surface-altering moiety can be determined. Using the molecular weight of the surface-altering moiety the number of surface-altering moiety to mass of core material can be calculated. To turn this number into a surface density measurement, the surface area per mass of core material needs to be calculated. The volume of the particle is approximated as that of a sphere with the diameter obtained from DLS allowing for the calculation of the surface area per mass of core material. In this way the number of surface-altering moieties per surface area can be determined.

An example of calculating this surface density is presented in Example 5 below using the surface area of a perfect sphere with the diameter of the core particles determined by dynamic light scattering. In alternative embodiments surface area is measured as the Brunauer-Emmett-Teller specific surface area which is based on the adsorption of gas molecules to solid surfaces. Most typically nitrogen is the gas used.

In certain embodiments in which the surface-altering agent is adsorbed onto a surface of a core, the surface-altering agent may be in equilibrium with other molecules of the surface-altering agent in solution. In some cases, the adsorbed surface-altering agent may be present on the surface of the core at a density described herein.

A coating comprising a surface-altering agent may partially or completely surround the core. For example, the coating may surround at least about 10%, at least about 30%, at least about 50%, at least about 60%, at least about 70%, at least about 80%, at least about 90%, at least about 99%, up to about 100%, up to about 90%, up to about 80%, up to about 70%, up to about 60%, or up to about 50%, about 80-100% of the surface area of a core, or any percentage in a range bounded by any of these values.

A coating of a particle can have any suitable thickness. For example, a coating may have an average thickness of at least about 1 nm, about 5 nm, about 10 nm, about 30 nm, about 50 nm, about 100 nm, about 200 nm, about 500 nm, about 1 μm, or about 5 μm. In other embodiments, the coating may have an average thickness of up to about 5 μm, about 1 μm, about 500 nm, about 200 nm, about 100 nm, about 50 nm, about 30 nm, about 10 nm, or about 5 nm. In other embodiments, the coating may have an average thickness of about 1-100 nm, or any thickness in a range bounded by any of the preceding values. Thickness is determined by comparison of particle sizes of the coated particle and the corresponding uncoated core particle using dynamic light scattering.

In some embodiments, two or more surface-altering agents, such as two or more of a PEG-PPO-PEG triblock copolymer, a synthetic polymer having pendant OH groups (e.g. PVA), and a polysorbate, may be present in the coating. Furthermore, although many of the embodiments described herein involve a single coating, in other embodiments, a particle may include more than one coating (e.g., at least two, three, four, five, or more coatings), and each coating need not be formed of, or comprise, a mucus penetrating material. In some cases, an intermediate coating (i.e., a coating between the core surface and an outer coating) may include a polymer that facilitates attachment of an outer coating to the core surface. In many embodiments, an outer coating of a particle includes a polymer comprising a material that facilitates the transport of the particle through mucus.

### Pharmaceutical Formulations

A subject composition may optionally comprise ophthalmically acceptable carriers, additives, diluents, or a combination thereof. For ophthalmic application, solutions or medicaments may be prepared using a physiological saline solution as a carrier or diluent. Ophthalmic solutions may be maintained at a physiologic pH with an appropriate buffer system. The formulations may also contain conventional additives, such as pharmaceutically acceptable buffers, preservatives, stabilizers and surfactants.

Pharmaceutical compositions described herein and for use in accordance with the articles and methods described herein may include a pharmaceutically acceptable excipient or carrier. A pharmaceutically acceptable excipient or pharmaceutically acceptable carrier may include a non-toxic, inert solid, semi-solid or liquid filler, diluent, encapsulating material or formulation auxiliary of any suitable type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on the route of administration as described below, the pharmaceutical agent being delivered, time course of delivery of the agent, etc.

A subject composition may include one or more buffers. Examples include, but are not limited to, acetate buffers, citrate buffers, phosphate buffers, borate buffers, lactate buffers, NaOH/Trolamine buffers, or a combination thereof such as phosphate and citrate or borate and citrate. Acids or bases, such as HCl and NaOH, may be used to adjust the pH of these formulations as needed. The amount of buffer used may vary. In some embodiments, the buffer may have a concentration in a range of about 1 nM to about 100 mM.

A subject composition may include one or more preservatives. The preservatives may vary, and may include any compound or substance suitable for reducing or preventing microbial contamination in an ophthalmic liquid subject to multiple uses from the same container. Preservatives that may be used in the pharmaceutical compositions disclosed herein include, but are not limited to, cationic preservatives such as quaternary ammonium compounds including benzalkonium chloride, polyquaternium-1 (Polyquad®), and the like; guanidine-based preservatives including PHMB, chlorhexidine, and the like; chlorobutanol; mercury preservatives such as thimerosal, phenylmercuric acetate and phenylmercuric nitrate; and other preservatives such as benzyl alcohol. In some embodiments, a preservative may have a concentration of about 10 ppm to about 200 ppm, about 10 ppm to about 300 ppm, or about 50 ppm to about 150 ppm.

A subject composition may include one or more surfactants of the following classes: alcohols; amine oxides; block polymers; carboxylated alcohol or alkylphenol ethoxylates; carboxylic acids/fatty acids; ethoxylated alcohols; ethoxylated alkylphenols; ethoxylated aryl phenols; ethoxylated fatty acids; ethoxylated; fatty esters or oils (animal & veg.); fatty esters; fatty acid methyl ester ethoxylates; glycerol esters; glycol esters; lanolin-based derivatives; lecithin and lecithin derivatives; lignin and lignin derivatives; methyl esters; monoglycerides and derivatives; polyethylene glycols; polymeric surfactants; propoxylated & ethoxylated fatty acids, alcohols, or alkyl phenols; protein-based surfactants; sarcosine derivatives; sorbitan derivatives; sucrose and glucose esters and derivatives. The amount of surfactant may vary. In some embodiments, the amount of any surfactant such as those listed above may be about 0.001 to about 5%, about 0.1% to about 2%, or about 0.1% to about 1%.

A subject composition may include one or more tonicity adjusters. The tonicity adjusters may vary, and may include any compound or substance useful for adjusting the tonicity of an ophthalmic liquid. Examples include, but are not limited to, salts, particularly sodium chloride or potassium chloride, organic compounds such as propylene glycol, mannitol, or glycerin, or any other suitable ophthalmically acceptable tonicity adjustor. The amount of tonicity adjuster may vary depending upon whether an isotonic, hypertonic, or hypotonic liquid is desired. In some embodiments, the amount of a tonicity agent such as those listed above may be at least about 0.0001% up to about 1%, about 2%, or about 5%. In some embodiments a subject composition comprises glycerin.

The osmolality of a subject composition may be hypotonic, isotonic, or hypertonic. For example, a subject composition may have an osmolarity of about 200-250 mOsm/kg, about 250-280 mOsm/kg, about 280-320 mOsm/kg, about 290-310 mOsm/kg, about 295-305 mOsm/kg, about 300 mOsm/kg (isotonic), about 300-350 mOsm/kg, or any osmolarity in a range bounded by any of these values. To achieve a formulation of an osmolarity of about 300 mOsm/kg, the concentration of sodium chloride in the formulation is typically about 0.9%. A combination of 1.2% glycerin and 0.45% sodium chloride generally also yields an isotonic solution.

A subject composition may include an antioxidant such as sodium metabisulfite, sodium thiosulfate, acetylcysteine, butylated hydroxyanisole, and butylated hydroxytoluene.

A subject composition may include a chelating agent such as edetate disodium.

A subject composition may be suitable for administration to an eye, such as topical administration to the eye or direct injection into the eye.

Generally, it is desirable for a drug to be pure. For example, it should contain low levels of impurities, such as degradants formed during sterilization or other processing steps, or formed over time during storage. In some embodiments, the level of any degradant of the pharmaceutical agent, such as a hydrocortisone derivative disclosed herein, is no more than about 1 wt %, about 0.9 wt %, about 0.8 wt %, about 0.7 wt %, about 0.6 wt %, about 0.5 wt %, about 0.4 wt %, about 0.3 wt %, about 0.2 wt %, about 0.15 wt %, about 0.1 wt %, about 0.03 wt %, about 0.01 wt %, about 0.003 wt %, or about 0.001 wt % relative to the weight of the pharmaceutical agent.

A subject composition may be administered by any suitable route, such as orally in any acceptable form (e.g., tablet, liquid, capsule, powder, and the like); topically in any acceptable form (e.g., patch, eye drops, creams, gels, nebulization, punctal plug, drug eluting contact, iontophoresis, and ointments); by injection in any acceptable form (e.g., periocular, intravenous, intraperitoneal, intramuscular, subcutaneous, parenteral, and epidural); by inhalation; and by implant or the use of reservoirs (e.g., subcutaneous pump, intrathecal pump, suppository, biodegradable delivery system, non-biodegradable delivery system and other implanted extended or slow release device or formulation). The target may be the eye or another organ or tissue. In some embodiments, a subject composition is administered to an eye in order to deliver the pharmaceutical agent to a tissue in the eye of the subject.

A subject composition may be administered at any suitable frequency. For example, two or more doses of a subject composition may be administered to subject, e.g. to an eye of a subject, wherein the period between consecutive doses is at least about 4 hours, at least about 6 hours, at least about 8 hours, at least about 12 hours, at least about 24 hours, at least about 36 hours, or at least about 48 hours, at least a week, or at least a month.

A subject composition may be administered to treat, diagnose, prevent, or manage a disease or condition in a subject, including a human being or a non-human animal, such as a mammal. In some embodiments, the condition is an ocular condition, such as condition affecting the anterior or front of the eye, such as post-surgical inflammation, uveitis, infections, aphakia, pseudophakia, astigmatism, blepharospasm, cataract, conjunctival diseases, conjunctivitis, corneal diseases, corneal ulcer, dry eye syndromes, eyelid diseases, lacrimal apparatus diseases, lacrimal duct obstruction, myopia, presbyopia; pupil disorders, corneal neovascularization; refractive disorders, and strabismus. Glaucoma can be considered to be a front of the eye ocular condition in some embodiments because a clinical goal of glaucoma treatment can be to reduce a hypertension of aqueous fluid in the anterior chamber of the eye (i.e., reduce intraocular pressure).

The leading causes of vision impairment and blindness are conditions linked to the posterior segment of the eye. These conditions may include, without limitation, age-related ocular degenerative diseases such as, macular degeneration, including acute macular degeneration, exudative and non-exudative age related macular degeneration (collectively AMD), proliferative vitreoretinopathy (PVR), retinal ocular condition, retinal damage, macular edema (e.g., cystoid macular edema (CME) or (diabetic macular edema (DME)), endophthalmitis; intraocular melanoma; acute macular neuroretinopathy; Behcet's disease; choroidal neovascularization; uveitis; diabetic uveitis; histoplasmosis; infections, such as fungal or viral-caused infections; edema; multifocal choroiditis; ocular trauma which affects a posterior ocular site or location; ocular tumors; retinal disorders, such as central retinal vein occlusion, diabetic retinopathy (including proliferative diabetic retinopathy), retinal arterial occlusive disease, retinal detachment, uveitic retinal disease; sympathetic opthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, retinoblastoma. Glaucoma can be considered a posterior ocular condition in some embodiments because the therapeutic goal is to prevent the loss of or reduce the occurrence of loss of vision due to damage to or loss of retinal cells or optic nerve cells (i.e., neuroprotection). In fact, certain forms of glaucoma are not characterized by high IOP, but mainly by retinal degeneration alone.

Some embodiments include administering a subject composition to treat inflammation, macular degeneration, macular edema, uveitis, dry eye, or glaucoma.

### Preparation of Coated Particles

While there are many potential ways to coat drug or core particles with a surface-altering agent, typically this could involve milling the particles (such as drug particles) with a surface-altering agent or incubating particles in an aqueous solution in the presence of a surface-altering agent. Another useful method involves dissolving a drug in an organic solvent and emulsifying the solution in water using the surface-altering agent as a surfactant, then removing the organic solvent by evaporation (e.g. by rotary evaporation). Combinations of these methods may also be used.

In a wet milling process, milling can be performed in a dispersion (e.g., an aqueous dispersion) containing one or more surface-altering agents, a grinding medium, a solid to be milled (e.g., a solid pharmaceutical agent), and a solvent. Any suitable amount of a surface-altering agent can be included in the solvent. In some embodiments, a surface-altering agent may be present in the solvent in an amount of at least about 0.001% (wt % or weight to volume (w:v)), at least about 0.01%, at least about 0.1%, at least about 0.5%, at least about 1%, at least about 2%, at least about 3%, at least about 4%, at least about 5%, at least about 6%, at least about 7%, at least about 8%, at least about 10%, at least about 12%, at least about 15%, at least about 20%, at least about 40%, at least about 60%, or at least about 80% of the solvent. In some cases, the surface-altering agent may be present in the solvent in an amount of about 100% (e.g., in an instance where the surface-altering agent is the solvent). In other embodiments, the surface-altering agent may be present in the solvent in an amount of less than or equal to about 100%, less than or equal to about 80%, less than or equal to about 60%, less than or equal to about 40%, less than or equal to about 20%, less than or equal to about 15%, less than or equal to about 12%, less than or equal to about 10%, less than or equal to about 8%, less than or equal to about 7%, less than or equal to about 6%, less than or equal to about 5%, less than or equal to about 4%, less than or equal to about 3%, less than or equal to about 2%, or less than or equal to about 1% of the solvent. Combinations of the above-referenced ranges are also possible (e.g., an amount of less than or equal to about 5% and at least about 1% of the solvent). Other ranges are also possible. In certain embodiments, the surface-altering agent is present in the solvent in an amount of about 0.01-2% of the solvent. In certain embodiments, the surface-altering agent is present in the solvent in an amount of about 0.2-20% of the solvent. In certain embodiments, the surface-altering agent is present in the solvent in an amount of about 0.1% of the solvent. In certain embodiments, the surface-altering agent is present in the solvent in an amount of about 0.4% of the solvent. In certain embodiments, the surface-altering agent is present in the solvent in an amount of about 1% of the solvent. In certain embodiments, the surface-altering agent is present in the solvent in an amount of about 2% of the solvent. In certain embodiments, the surface-altering agent is present in the solvent in an amount of about 5% of the solvent. In certain embodiments, the surface-altering agent is present in the solvent in an amount of about 10% of the solvent.

The particular range chosen may influence factors that may affect the ability of the particles to penetrate mucus such as the stability of the coating of the surface-altering agent on the particle surface, the average thickness of the coating of the surface-altering agent on the particles, the orientation of the surface-altering agent on the particles, the density of the surface altering agent on the particles, surface-altering agent:drug ratio, drug concentration, the size, dispersibility, and polydispersity of the particles formed, and the morphology of the particles formed.

The pharmaceutical agent may be present in the solvent in any suitable amount. In some embodiments, the pharmaceutical agent is present in an amount of at least about 0.001% (wt % or % weight to volume (w:v)), at least about 0.01%, at least about 0.1%, at least about 0.5%, at least about 1%, at least about 2%, at least about 3%, at least about 4%, at least about 5%, at least about 6%, at least about 7%, at least about 8%, at least about 10%, at least about 12%, at least about 15%, at least about 20%, at least about 40%, at least about 60%, or at least about 80% of the solvent. In some cases, the pharmaceutical agent may be present in the solvent in an amount of less than or equal to about 100%, less than or equal to about 90%, less than or equal to about 80%, less than or equal to about 60%, less than or equal to about 40%, less than or equal to about 20%, less than or equal to about 15%, less than or equal to about 12%, less than or equal to about 10%, less than or equal to about 8%, less than or equal to about 7%, less than or equal to about 6%, less than or equal to about 5%, less than or equal to about 4%, less than or equal to about 3%, less than or equal to about 2%, or less than or equal to about 1% of the solvent. Combinations of the above-referenced ranges are also possible (e.g., an amount of less than or equal to about 20% and at least about 1% of the solvent). In some embodiments, the pharmaceutical agent is present in the above ranges but in w:v

The ratio of surface-altering agent to pharmaceutical agent in a solvent may also vary. In some embodiments, the ratio of surface-altering agent to pharmaceutical agent may be at least 0.001:1 (weight ratio, molar ratio, or w:v ratio), at least 0.01:1, at least 0.01:1, at least 1:1, at least 2:1, at least 3:1, at least 5:1, at least 10:1, at least 25:1, at least 50:1, at least 100:1, or at least 500:1. In some cases, the ratio of surface-altering agent to pharmaceutical agent may be less than or equal to 1000:1 (weight ratio or molar ratio), less than or equal to 500:1, less than or equal to 100:1, less than or equal to 75:1, less than or equal to 50:1, less than or equal to 25:1, less than or equal to 10:1, less than or equal to 5:1, less than or equal to 3:1, less than or equal to 2:1, less than or equal to 1:1, or less than or equal to 0.1:1. Combinations of the above-referenced ranges are possible (e.g., a ratio of at least 5:1 and less than or equal to 50:1). Other ranges are also possible.

It should be appreciated that while in some embodiments the stabilizer used for milling forms a coating on a particle surface, which coating renders particle mucus penetrating, in other embodiments, the stabilizer may be exchanged with one or more other surface-altering agents after the particle has been formed. For example, in one set of methods, a first stabilizer/surface-altering agent may be used during a milling process and may coat a surface of a core particle, and then all or portions of the first stabilizer/surface-altering agent may be exchanged with a second stabilizer/surface-altering agent to coat all or portions of the core particle surface. In some cases, the second stabilizer/surface-altering agent may render the particle mucus penetrating more than the first stabilizer/surface-altering agent. In some embodiments, a core particle having a coating including multiple surface-altering agents may be formed.

Any suitable grinding medium can be used for milling. In some embodiments, a ceramic and/or polymeric material and/or a metal can be used. Examples of suitable materials may include zirconium oxide, silicon carbide, silicon oxide, silicon nitride, zirconium silicate, yttrium oxide, glass, alumina, alpha-alumina, aluminum oxide, polystyrene, poly(methyl methacrylate), titanium, steel. A grinding medium may have any suitable size. For example, the grinding medium may have an average diameter of at least about 0.1 mm, at least about 0.2 mm, at least about 0.5 mm, at least about 0.8 mm, at least about 1 mm, at least about 2 mm, or at least about 5 mm. In some cases, the grinding medium may have an average diameter of less than or equal to about 5 mm, less than or equal to about 2 mm, less than or equal to about 1 mm, less than or equal to about 0.8, less than or equal to about 0.5 mm, or less than or equal to about 0.2 mm. Combinations of the above-referenced ranges are also possible (e.g., an average diameter of at least about 0.5 millimeters and less than or equal to about 1 mm). Other ranges are also possible.

Any suitable solvent may be used for milling. The choice of solvent may depend on factors such as the solid material (e.g., pharmaceutical agent) being milled, the particular type of stabilizer/surface-altering agent being used (e.g., one that may render the particle mucus penetrating), the grinding material be used, among other factors. Suitable solvents may be ones that do not substantially dissolve the solid material or the grinding material, but dissolve the stabilizer/surface-altering agent to a suitable degree. Non-limiting examples of solvents may include water, buffered solutions, other aqueous solutions, alcohols (e.g., ethanol, methanol, butanol), and mixtures thereof that may optionally include other components such as pharmaceutical excipients, polymers, pharmaceutical agents, salts, preservative agents, viscosity modifiers, tonicity modifier, taste masking agents, antioxidants, pH modifier, and other pharmaceutical excipients. In other embodiments, an organic solvent can be used.

The following embodiments are contemplated:

### Embodiment 1

A pharmaceutical composition suitable for administration to an eye, comprising: a plurality of coated particles, comprising: a core particle comprising a hydrocortisone derivative is

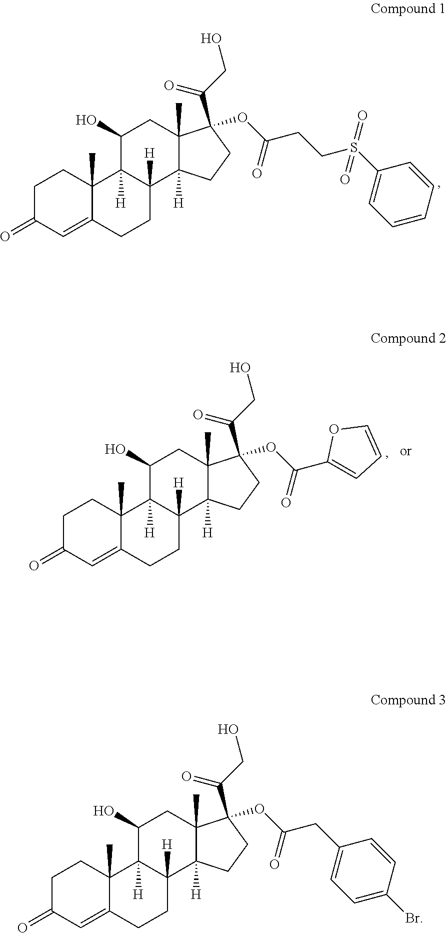

and a mucus penetration-enhancing coating comprising a surface-altering agent surrounding the core particle, wherein the surface-altering agent comprises one or more of the following components: a) a triblock copolymer comprising a hydrophilic block-hydrophobic block-hydrophilic block configuration, wherein the hydrophobic block has a molecular weight of at least about 2 kDa, and the hydrophilic blocks constitute at least about 15 wt % of the triblock copolymer, wherein the hydrophobic block associates with the surface of the core particle, and wherein the hydrophilic block is present at the surface of the coated particle and renders the coated particle hydrophilic, b) a synthetic polymer having pendant hydroxyl groups on the backbone of the polymer, the polymer having a molecular weight of at least about 1 kDa and less than or equal to about 1000 kDa, wherein the polymer is at least about 30% hydrolyzed and less than about 95% hydrolyzed, or c) a polysorbate, wherein the surface altering agent is present on the outer surface of the core particle at a density of at least 0.01 molecules/nm², wherein the surface altering agent is present in the pharmaceutical composition in an amount of between about 0.001% to about 5% by weight; and an ophthalmically acceptable carrier, additive, or diluent.

### Embodiment 2

A pharmaceutical composition suitable for treating an ocular disorder by administration to an eye, comprising: a plurality of coated particles, comprising: a core particle comprising a hydrocortisone derivative selected from Compounds 1, 2, and 3, and a mucus penetration-enhancing coating comprising a surface-altering agent surrounding the core particle, wherein the surface-altering agent comprises one or more of the following components: a) a triblock copolymer comprising a hydrophilic block-hydrophobic block-hydrophilic block configuration, wherein the hydrophobic block has a molecular weight of at least about 2 kDa, and the hydrophilic blocks constitute at least about 15 wt % of the triblock copolymer, b) a synthetic polymer having pendant hydroxyl groups on the backbone of the polymer, the polymer having a molecular weight of at least about 1 kDa and less than or equal to about 1000 kDa, wherein the polymer is at least about 30% hydrolyzed and less than about 95% hydrolyzed, or c) a polysorbate, wherein the plurality of coated particles have an average smallest cross-sectional dimension of less than about 1 micron; and wherein the coating on the core particle is present in a sufficient amount to increase the concentration of the hydrocortisone derivative and/or hydrocortisone metabolite in a cornea or an aqueous humor after administration when administered to the eye, compared to the concentration of the hydrocortisone derivative in the cornea or the aqueous humor when administered as a core particle without the coating.

### Embodiment 3

The pharmaceutical composition according to embodiments 1 or 2 wherein the hydrocortisone derivative is (10R,11S,13S,17R)-11-hydroxy-17-(2-hydroxyacetyl)-10,13-dimethyl-3-oxo-2,3,6,7,8,9,10,11,12,13,14,15,16,17-tetradecahydro-1H-cyclopenta[a]phenanthren-17-yl 3-(phenylsulfonyl)propanoate.

### Embodiment 4

The pharmaceutical composition according to embodiments 1 or 2 wherein the hydrocortisone derivative is (10R,11S,13S,17R)-11-hydroxy-17-(2-hydroxyacetyl)-10,13-dimethyl-3-oxo-2,3,6,7,8,9,10,11,12,13,14,15,16,17-tetradecahydro-1H-cyclopenta[a]phenanthren-17-ylfuran-2-carboxylate

### Embodiment 5

The pharmaceutical composition according to embodiments 1 or 2 wherein the hydrocortisone derivative is (10R,11S,13S,17R)-11-hydroxy-17-(2-hydroxyacetyl)-10,13-dimethyl-3-oxo-2,3,6,7,8,9,10,11,12,13,14,15,16,17-tetradecahydro-1H-cyclopenta[a]phenanthren-17-yl 2-(4-bromophenyl)acetate.

### Embodiment 6

The pharmaceutical composition of embodiment 1 or 2, wherein the hydrocortisone derivative is Compound 1:

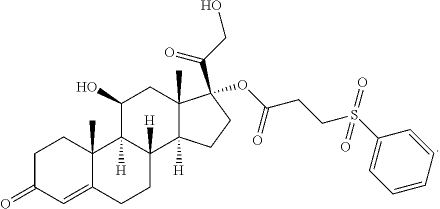

### Embodiment 7

The pharmaceutical composition of embodiment 6, wherein Compound 1 is in crystalline form B having XRPD peaks at 5.88, 10.36, 13.18, 14.40, 15.55, 17.57, and 20.82±0.2° 2θ.

### Embodiment 8

The pharmaceutical composition of embodiment 1 or 2, wherein the hydrocortisone derivative is Compound 2:

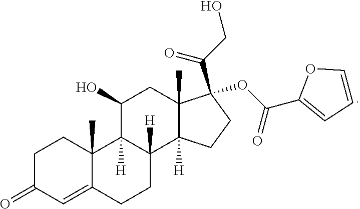

### Embodiment 9

The pharmaceutical composition of embodiment 8, wherein Compound 2 is in crystalline form A having XRPD peaks at 5.83, 10.09, 11.72, 14.49, 15.32, and 15.66±0.2° 2θ.

### Embodiment 10

The pharmaceutical composition of embodiment 1 or 2, wherein the hydrocortisone derivative is Compound 3:

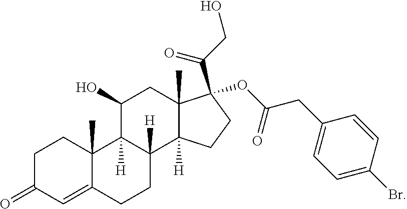

### Embodiment 11

The pharmaceutical composition of claim 10, wherein Compound 3 is in crystalline form A having XRPD peaks at 5.08, 7.18, 13.90, and 20.45±0.2° 2θ.

### Embodiment 12

The pharmaceutical composition of claim 10, wherein Compound 3 is in crystalline form B having XRPD peaks at 8.88, 12.66, 14.34, 19.02, 20.28, 20.63 and 25.71±0.2° 2θ.

### Embodiment 13

The pharmaceutical composition of any one of embodiments 1-12, wherein the surface-altering agent is covalently attached to the core particles.

### Embodiment 14

The pharmaceutical composition of any one of embodiments 1-12, wherein the surface-altering agent is non-covalently adsorbed to the core particles.

### Embodiment 15

The pharmaceutical composition of any one of embodiments 1-14, wherein the surface-altering agent is present on the surfaces of the coated particles at a density of at least about 0.1 molecules per nanometer squared.

### Embodiment 16

The pharmaceutical composition of any one of embodiments 1-12, wherein the surface-altering agent comprises the triblock copolymer.

### Embodiment 17

The pharmaceutical composition of any one of embodiments 1-12, wherein the surface-altering agent comprises the triblock copolymer, wherein the hydrophilic blocks of the triblock copolymer constitute at least about 30 wt % of the triblock polymer and less than or equal to about 80 wt % of the triblock copolymer.

### Embodiment 18

The pharmaceutical composition of embodiment 16 or 17, wherein the hydrophobic block portion of the triblock copolymer has a molecular weight of about 3 kDa to about 8 kDa.

### Embodiment 19

The pharmaceutical composition of any one of embodiments 16-18, wherein the triblock copolymer is poly(ethylene oxide)-poly(propylene oxide)-poly(ethylene oxide).

### Embodiment 20

The pharmaceutical composition of any one of embodiments 1-12, wherein the surface-altering agent comprises a linear polymer having pendant hydroxyl groups on the backbone of the polymer.

### Embodiment 21

The pharmaceutical composition of any one of embodiments 1-20, wherein the surface-altering agent has a molecular weight of at least about 4 kDa.

### Embodiment 22

The pharmaceutical composition of any one of embodiments 1-12, wherein the surface altering agent is polyvinyl alcohol.

### Embodiment 23

The pharmaceutical composition of embodiment 22, wherein the poly(vinyl alcohol) is about 70% to about 94% hydrolyzed.

### Embodiment 24

The pharmaceutical composition of any one of embodiments 1-23, wherein the hydrocortisone derivative is crystalline.

### Embodiment 25

The pharmaceutical composition of any one of embodiments 1-23, wherein the hydrocortisone derivative is amorphous.

### Embodiment 26

The pharmaceutical composition of any one of embodiments 1-25, wherein the core particles comprise a hydrocortisone derivative that is encapsulated in a polymer, a lipid, a protein, or a combination thereof.

### Embodiment 27

The pharmaceutical composition of any one of embodiments 1-26, wherein the hydrocortisone derivative constitutes at least about 80 wt % of the core particle.

### Embodiment 28

The pharmaceutical composition of any one of embodiments 1-27, wherein the coated particles have an average size of about 10 nm to about 1 μm.

### Embodiment 29

The pharmaceutical composition of any one of embodiments 1-28, comprising one or more degradants of the hydrocortisone derivative, and wherein the concentration of each degradant is 0.1 wt % or less relative to the weight of the hydrocortisone.

### Embodiment 30

The pharmaceutical composition of any one of embodiments 1-29, wherein the polydispersity index of the composition is less than or equal to about 0.5.

### Embodiment 31

The pharmaceutical composition of any one of embodiments 1-30, wherein the pharmaceutical composition is suitable for topical administration to the eye.

### Embodiment 32

The pharmaceutical composition of any one of embodiments 1-31, wherein the pharmaceutical composition is suitable for direct injection into the eye.

### Embodiment 33

The pharmaceutical composition of any one of embodiments 1-32, wherein the ophthalmically acceptable carrier, additive, or diluent comprises glycerin.

### Embodiment 34

A method of treating, diagnosing, preventing, or managing an ocular condition in a subject, the method comprising: administering a pharmaceutical composition of any one of embodiments 1-32 to an eye of a subject and thereby delivering the hydrocortisone derivative and/or hydrocortisone metabolite to a tissue in the eye of the subject.

### Embodiment 35

The method of embodiment 34, wherein after administering the pharmaceutical composition topically to the eye, an ophthalmically efficacious level of the hydrocortisone derivative and/or its hydrocortisone metabolite are/is delivered to a palpebral conjunctiva, a bulbar conjunctiva, a fornix conjunctiva, an aqueous humor, an anterior sclera, or a cornea for at least 12 hours after administration.

### Embodiment 36

The method of any one of embodiments 34 or 35, wherein the hydrocortisone derivative and/or its hydrocortisone metabolite are/is delivered to a tissue in the front of the eye of the subject.

### Embodiment 37

The method of embodiment 34, wherein the hydrocortisone derivative and/or its hydrocortisone metabolite are/is delivered to a tissue in the back of the eye of the subject.

### Embodiment 38

The method of embodiment 34, wherein the tissue is a retina, a macula, a posterior sclera, vitreous humor, or a choroid.

### Embodiment 39

The method of any one of embodiments 34-38, wherein the ocular condition is inflammation, macular degeneration, macular edema, uveitis, glaucoma, or dry eye.

### EXAMPLES

### Example 1

The following describes a non-limiting example of a method of forming non-polymeric solid particles into mucus-penetrating particles. Pyrene, a hydrophobic naturally fluorescent compound, was used as the core particle and was prepared by a milling process in the presence of various surface-altering agents. The surface-altering agents formed coatings around the core particles. Different surface-altering agents were evaluated to determine effectiveness of the coated particles in penetrating mucus.

Pyrene was milled in aqueous dispersions in the presence of various surface-altering agents to determine whether certain surface-altering agents can: 1) aid particle size reduction to several hundreds of nanometers and 2) physically (non-covalently) coat the surface of generated nanoparticles with a coating that would minimize particle interactions with mucus constituents and prevent mucus adhesion. The surface-altering agents tested included a variety of polymers, oligomers, and small molecules listed in Table 3 below, including pharmaceutically relevant excipients such as poly(ethylene oxide)-poly(propylene oxide)-poly(ethylene oxide) block copolymers (Pluronic® copolymers), polyvinylpyrrolidones (Kollidon), and hydroxypropyl methylcellulose (Methocel), etc.

TABLE 3
Surface-altering agents tested with pyrene as a model compound.

| | | |
|---|---|---|
| | Acronym or | |
| Stabilizer | Trade Name | Grade or Molecular Weight |

Polymeric surface-altering agents

| | | |
|---|---|---|
| Poly(ethylene oxide)- | Pluronic ® | F127, F108, F68, F87, F38, P123, |
| poly(propylene oxide)- | | P105, P103, P65, L121, L101, L81, |
| poly(ethylene oxide) block | | L44, L31 |
| copolymers | | |
| Polyvinylpyrrolidone | PVP | Kollidon 17 (9K), Kollidon 25 (26K), |
| | | Kollindon 30 (43K) |
| PVA-poly(ethylene glycol) graft- | | Kollicoat IR |
| copolymer | | |
| Hydroxypropyl methylcellulose | HPMC | Methocel E50, Methocel K100 |

Oligomeric surface-altering agents
Tween 20
Tween 80
Solutol HS 15
Triton X100
Tyloxapol
Cremophor RH 40
Small molecule surface-altering agents
Span 20
Span 80
Octyl glucoside
Cetytrimethylammonium bromide (CTAB)
Sodium dodecyl sulfate (SDS)

An aqueous dispersion containing pyrene and one of the surface-altering agents listed above was milled with milling media until particle size was reduced below 500 nm. Table 4 lists particle size characteristics of pyrene particles obtained by milling in the presence of the various surface-altering agents. Particle size was measured by dynamic light scattering. When Pluronic® L101, L81, L44, L31, Span 20, Span 80, or Octyl glucoside were used as surface-altering agents, stable nanosuspensions could not be obtained. Therefore, these surface-altering agents were excluded from further investigation due to their inability to effectively aid particle size reduction.

TABLE 4
Particle size measured by DLS in nanosuspensions obtained
by milling of pyrene with various surface-altering agents.

| Stabilizer | N-Ave. D (nm) |
|---|---|
| Pluronic ® F127 | 239 |
| Pluronic ® F108 | 267 |
| Pluronic ® P105 | 303 |
| Pluronic ® P103 | 319 |
| Pluronic ® P123 | 348 |
| Pluronic ® L121 | 418 |
| Pluronic ® F68 | 353 |
| Pluronic ® P65 | 329 |
| Pluronic ® F87 | 342 |
| Pluronic ® F38 | 298 |
| Pluronic ® L101 | not measurable* |
| Pluronic ® L81 | not measurable* |
| Pluronic ® L44 | not measurable* |
| Pluronic ® L31 | not measurable* |
| PVA 13K | 314 |
| PVA 31K | 220 |
| PVA 85K | 236 |
| Kollicoat IR | 192 |
| Kollidon 17 (PVP 9K) | 163 |
| Kollidon 25 (PVP 26K) | 210 |
| Kollindon 30 (PVP 43K) | 185 |
| Methocel E50 | 160 |
| Methocel K100 | 216 |
| Tween 20 | 381 |
| Tween 80 | 322 |
| Solutol HS | 378 |
| Triton X100 | 305 |
| Tyloxapol | 234 |
| Cremophor RH40 | 373 |
| SDS | 377 |
| CTAB | 354 |
| Span 20 | not measurable* |
| Span 80 | not measurable* |
| Octyl glucoside | not measurable* |

*milling with Pluronic ® L101, L81, L44, L31, Span 20, Span 80, Octyl glucoside failed to effectively reduce pyrene particle size and produce stable nanosuspensions.

The mobility and distribution of pyrene nanoparticles from the produced nanosuspensions in human cervicovaginal mucus (CVM) were characterized using fluorescence microscopy and multiple particle tracking software. In a typical experiment, ≤0.5 uL of a nanosuspension (diluted if necessary to the surfactant concentration of ˜1%) was added to 20 μl of fresh CVM along with controls. Conventional nanoparticles (200 nm yellow-green fluorescent carbon/late-modified polystyrene microspheres from Invitrogen) were used as a negative control to confirm the barrier properties of the CVM samples. Red fluorescent polystyrene nanoparticles covalently coated with PEG 5 kDa were used as a positive control with well-established MPP behavior. Using a fluorescent microscope equipped with a CCD camera, 15 s movies were captured at a temporal resolution of 66.7 ms (15 frames/s) under 100× magnification from several areas within each sample for each type of particles: sample (pyrene), negative control, and positive control (natural blue fluorescence of pyrene allowed observing of pyrene nanoparticles separately from the controls). Next, using an image processing software, individual trajectories of multiple particles were measured over a time-scale of at least 3.335 s (50 frames). Resulting transport data are presented here in the form of trajectory-mean velocity Vₘₑₐₙ, i.e., velocity of an individual particle averaged over its trajectory, and ensemble-average velocity <Vₘₑₐₙ>, i.e., Vₘₑₐₙ averaged over an ensemble of particles. To enable easy comparison between different samples and normalize velocity data with respect to natural variability in penetrability of CVM samples, relative sample velocity <Vₘₑₐₙ>ᵣₑₗ, was determined according to the formula shown in Equation 1.

$$\text{<} {V}_{mean} {\text{>}}_{rel} = \frac{\text{<} {V}_{mean} {\text{>}}_{Sample} - \text{<} {V}_{mean} {\text{>}}_{Negative control}}{\text{<} {V}_{mean} {\text{>}}_{Positive control} - \text{<} {V}_{mean} {\text{>}}_{Negative control}} \qquad ( Equation 1 )$$

Prior to quantifying mobility of the produced pyrene nanoparticles, their spatial distribution in the mucus sample was assessed by microscopy at low magnifications (10×, 40×). It was found that pyrene/Methocel nanosuspensions did not achieve uniform distribution in CVM and strongly aggregated into domains much larger than the mucus mesh size (data not shown). Such aggregation is indicative of mucoadhesive behavior and effectively prevents mucus penetration. Therefore, further quantitative analysis of particle mobility was deemed unnecessary. Similarly to the positive control, all other tested pyrene/stabilizer systems achieved a fairly uniform distribution in CVM. Multiple particle tracking confirmed that in all tested samples, the negative controls were highly constrained, while the positive controls were highly mobile as demonstrated by <Vₘₑₐₙ> for the positive controls being significantly greater than those for the negative controls (Table 5).

TABLE 5
Ensemble-average velocity <Vₘₑₐₙ> (um/s) and relative sample velocity <Vₘₑₐₙ>ᵣₑₗ
for pyrene/stabilizer nanoparticles (sample) and controls in CVM.

| | Negative | | Positive | | | | Sample | |
|---|---|---|---|---|---|---|---|---|
| | Control | | Control | | Sample | | (relative) | |
| Stabilizer | <Vₘₑₐₙ> | SD | <Vₘₑₐₙ> | SD | <Vₘₑₐₙ> | SD | <Vₘₑₐₙ>ᵣₑₗ | SD |
| Pluronic ® F127 | 0.58 | 0.18 | 5.97 | 0.54 | 6.25 | 0.72 | 1.05 | 0.18 |
| Pluronic ® F108 | 0.43 | 0.64 | 5.04 | 1.88 | 4.99 | 1.47 | 0.99 | 0.55 |
| Pluronic ® P105 | 0.56 | 0.52 | 4.38 | 1.36 | 4.47 | 2.11 | 1.02 | 0.69 |
| Pluronic ® P103 | 0.58 | 0.77 | 4.5 | 2.01 | 4.24 | 1.95 | 0.93 | 0.74 |
| Pluronic ® P123 | 0.56 | 0.44 | 4.56 | 1.44 | 3.99 | 1.66 | 0.86 | 0.54 |
| Pluronic ® L121 | 0.42 | 0.3 | 4.27 | 2.04 | 0.81 | 0.51 | 0.10 | 0.16 |
| Pluronic ® F68 | 0.56 | 0.52 | 4.38 | 1.36 | 0.81 | 0.7 | 0.07 | 0.23 |
| Pluronic ® P65 | 0.26 | 0.25 | 4.52 | 2.15 | 0.53 | 0.56 | 0.06 | 0.15 |
| Pluronic ® F87 | 0.95 | 1.6 | 5.06 | 1.34 | 0.74 | 0.78 | −0.05 | −0.43 |
| Pluronic ® F38 | 0.26 | 0.1 | 5.73 | 0.84 | 0.54 | 0.29 | 0.05 | 0.06 |
| Kollicoat IR | 0.62 | 0.62 | 5.39 | 0.55 | 0.92 | 0.81 | 0.06 | 0.22 |
| Kollidon 17 | 1.69 | 1.8 | 5.43 | 0.98 | 0.82 | 0.59 | −0.23 | −0.52 |
| Kollidon 25 | 0.41 | 0.34 | 5.04 | 0.64 | 1.29 | 1.09 | 0.19 | 0.25 |
| Kollindon 30 | 0.4 | 0.2 | 4.28 | 0.57 | 0.35 | 0.11 | −0.01 | 0.06 |
| Methocel E50** | | | | | | | | |
| Methocel K100** | | | | | | | | |
| Tween 20 | 0.77 | 0.93 | 5.35 | 1.76 | 1.58 | 2.02 | 0.18 | 0.49 |
| Tween 80 | 0.46 | 0.34 | 3.35 | 1.89 | 0.94 | 0.5 | 0.17 | 0.24 |
| Solutol HS | 0.42 | 0.13 | 3.49 | 0.5 | 0.8 | 0.6 | 0.12 | 0.20 |
| Triton X100 | 0.26 | 0.13 | 4.06 | 1.11 | 0.61 | 0.19 | 0.09 | 0.07 |
| Tyloxapol | 0.5 | 0.5 | 3.94 | 0.58 | 0.42 | 0.23 | −0.02 | −0.16 |
| Cremophor RH40 | 0.48 | 0.21 | 3.2 | 0.97 | 0.49 | 0.24 | 0.00 | 0.12 |
| SDS | 0.3 | 0.12 | 5.99 | 0.84 | 0.34 | 0.15 | 0.01 | 0.03 |
| CTAB | 0.39 | 0.09 | 4.75 | 1.79 | 0.32 | 0.31 | −0.02 | −0.07 |

*Did not produce stable nanosuspensions, hence not mucus-penetrating (velocity in CVM not measured)
**Aggregated in CVM, hence not mucus-penetrating (velocity in CVM not measured)

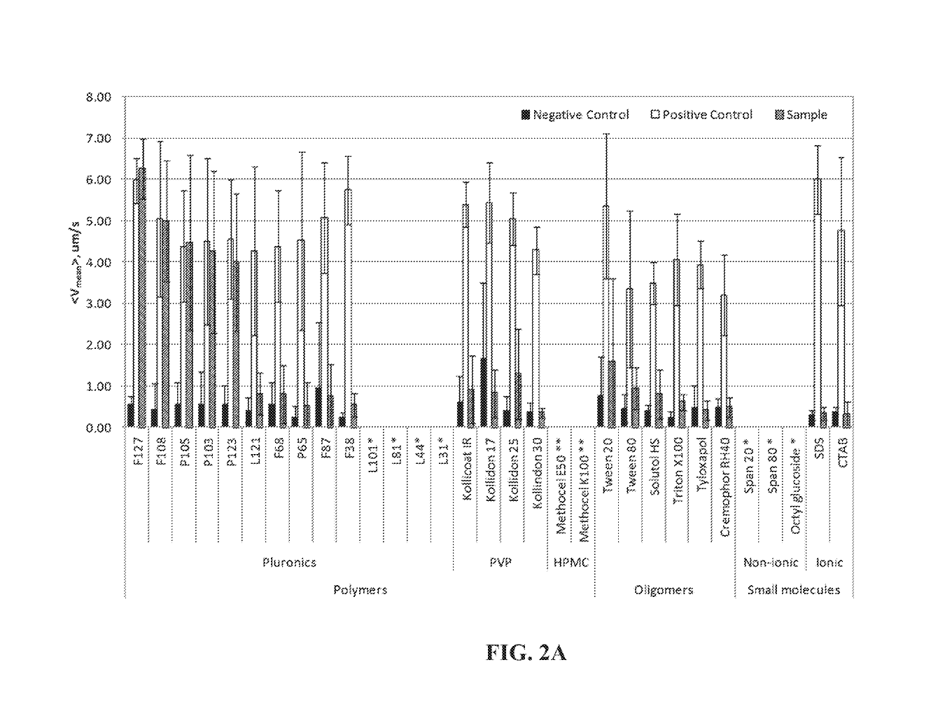

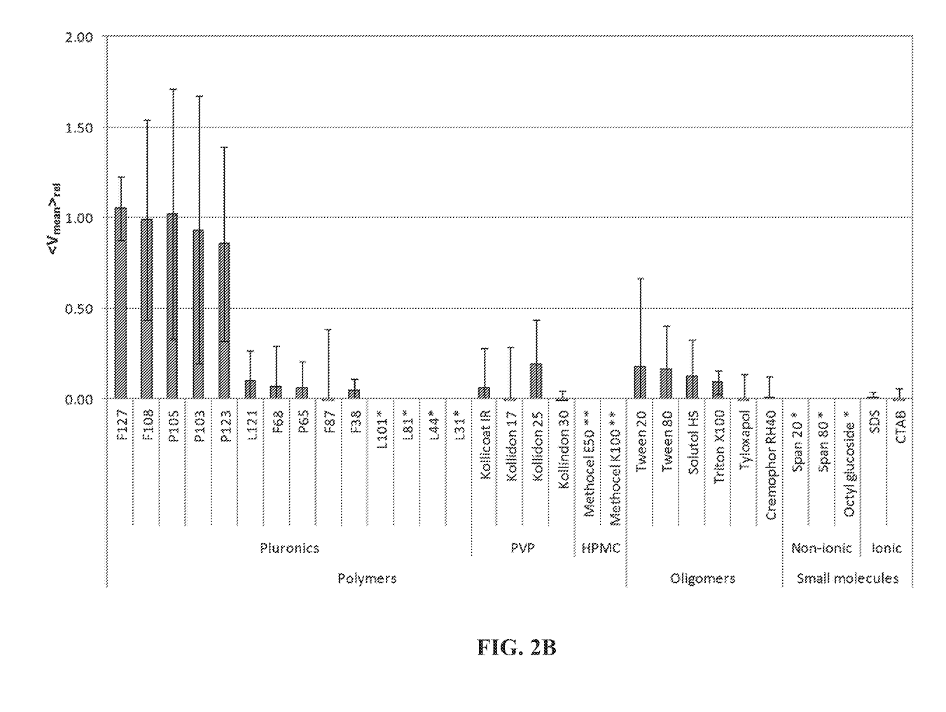

It was discovered that nanoparticles obtained in the presence of certain surface-altering agents migrate through CVM at the same rate or nearly the same velocity as the positive control. Specifically, pyrene nanoparticles stabilized with Pluronic® F127, F108, P123, P105, and P103 exhibited <Vₘₑₐₙ> that exceeded those of the negative controls by approximately an order of magnitude and were indistinguishable, within experimental error, from those of the positive controls, as shown in Table 5 and FIG. 2A. For these samples, <Vₘₑₐₙ>ᵣₑₗ values exceeded 0.5, as shown in FIG. 2B.

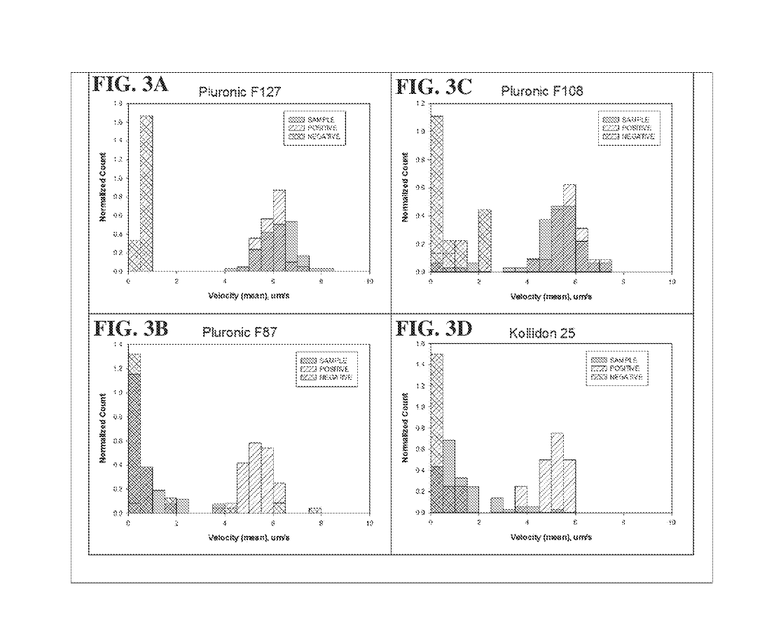

FIGS. 3A-3D are histograms showing distribution of Vₘₑₐₙ within an ensemble of particles. These histograms illustrate muco-diffusive behavior of samples stabilized with Pluronic® F127 and Pluronic® F108 (similar histograms were obtained for samples stabilized with Pluronic® P123, P105, and P103, but are not shown here) as opposed to muco-adhesive behavior of samples stabilized with Pluronic® 87, and Kollidon 25 (chosen as representative muco-adhesive samples).

To identify the characteristics of Pluronic® copolymers that render pyrene nanoparticles mucus penetrating, <Vₘₑₐₙ>ᵣₑₗ of the pyrene/Pluronic® nanoparticles was mapped with respect to molecular weight of the PPO block and the PEO weight content (%) of the Pluronic® copolymers used (FIG. 4). It was concluded that at least those Pluronic® copolymers that have the PPO block of at least 3 kDa and the PEO content of at least about 30 wt % rendered the nanoparticles mucus-penetrating.

### Example 2

The following describes a non-limiting example of a method of forming mucus-penetrating particles from pre-fabricated polymeric particles by physical adsorption of certain poly(vinyl alcohol) polymers (PVA). Carboxylated polystyrene nanoparticles (PSCOO) were used as the prefabricated particle/core particle with a well-established strongly mucoadhesive behavior. The PVAs acted as surface-altering agents forming coatings around the core particles. PVA of various molecular weights (MW) and hydrolysis degrees were evaluated to determine effectiveness of the coated particles in penetrating mucus.

PSCOO⁻ particles were incubated in aqueous solution in the presence of various PVA polymers to determine whether certain PVAs can physically (non-covalently) coat the core particle with a coating that would minimize particle interactions with mucus constituents and lead to rapid particle penetration in mucus. In these experiments, the PVA acted as a coating around the core particles, and the resulting particles were tested for their mobility in mucus, although in other embodiments, PVA may be exchanged with other surface-altering agents that can increase mobility of the particles in mucus. The PVAs tested ranged in the average molecular weight from 2 kDa to 130 kDa and in the average hydrolysis degree from 75% to 99+%. The PVAs that were tested are listed in Table 2, shown above.

The particle modification process was as follows: 200 nm red fluorescent PSCOO⁻ were purchased from Invitrogen. The PSCOO⁻ particles (0.4-0.5 wt %) were incubated in an aqueous PVA solution (0.4-0.5 wt %) for at least 1 hour at room temperature.

The mobility and distribution of the modified nanoparticles in CVM were characterized using fluorescence microscopy and multiple particle tracking software in a method similar to that described above. Multiple particle tracking confirmed that in all tested CVM samples the negative controls were constrained, while the positive controls were mobile as demonstrated by the differences in <Vₘₑₐₙ> for the positive and negative controls (Table 6).

TABLE 6
Transport of nanoparticles incubated with various PVA (sample) and controls in
CVM: Ensemble-average velocity <Vₘₑₐₙ> (μm/s) and relative sample velocity <Vₘₑₐₙ>ᵣₑₗ.

| | Negative | | Positive | | | | Sample | |
|---|---|---|---|---|---|---|---|---|
| | Control | | Control | | Sample | | (relative) | |
| Stabilizer | <Vₘₑₐₙ> | SD | <Vₘₑₐₙ> | SD | <Vₘₑₐₙ> | SD | <Vₘₑₐₙ>ᵣₑₗ | SD |
| PVA2K75 | 1.39 | 0.33 | 3.3 | 0.68 | 3.44 | 0.7 | 1.07 | 0.59 |
| PVA9K80 | 0.4 | 0.08 | 5.13 | 1.16 | 4.88 | 1.74 | 0.95 | 0.44 |
| PVA13K87 | 0.56 | 0.61 | 5.23 | 1.24 | 4.92 | 1.77 | 0.93 | 0.49 |
| PVA31K87 | 0.53 | 0.63 | 4.48 | 1.38 | 3.69 | 1.94 | 0.80 | 0.60 |
| PVA57K86 | 0.5 | 0.25 | 5.74 | 1.11 | 4.76 | 0.91 | 0.81 | 0.25 |
| PVA85K87 | 0.29 | 0.28 | 4.25 | 0.97 | 4.01 | 0.71 | 0.94 | 0.31 |
| PVA105K80 | 0.98 | 0.52 | 5.44 | 0.86 | 4.93 | 0.66 | 0.89 | 0.27 |
| PVA130K87 | 1.41 | 0.56 | 3.75 | 0.82 | 3.57 | 0.6 | 0.92 | 0.53 |
| PVA95K95 | 0.51 | 0.36 | 3.19 | 0.68 | 0.45 | 0.19 | −0.02 | −0.15 |
| PVA13K98 | 0.43 | 0.17 | 3.42 | 1.65 | 0.5 | 0.76 | 0.02 | 0.26 |
| PVA31K98 | 0.41 | 0.23 | 6.03 | 1.19 | 0.26 | 0.14 | −0.03 | −0.05 |
| PVA85K99 | 0.28 | 0.1 | 4.7 | 0.82 | 0.53 | 0.77 | 0.06 | 0.18 |

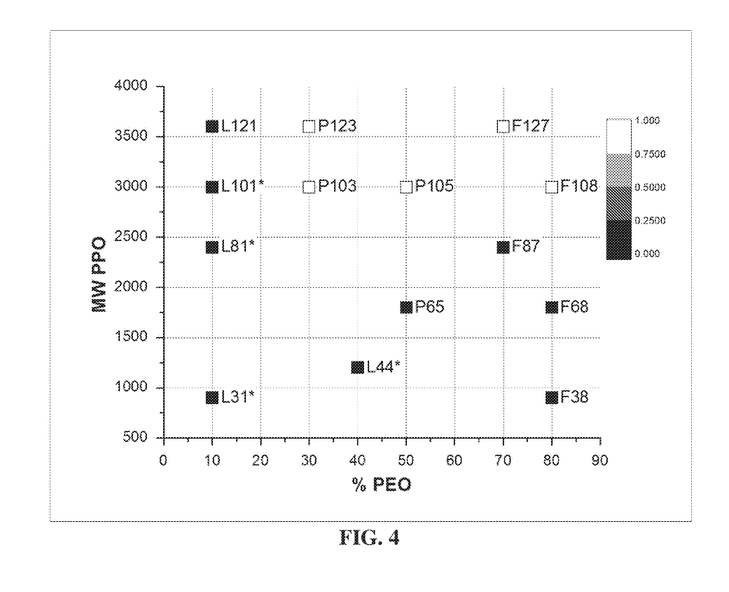

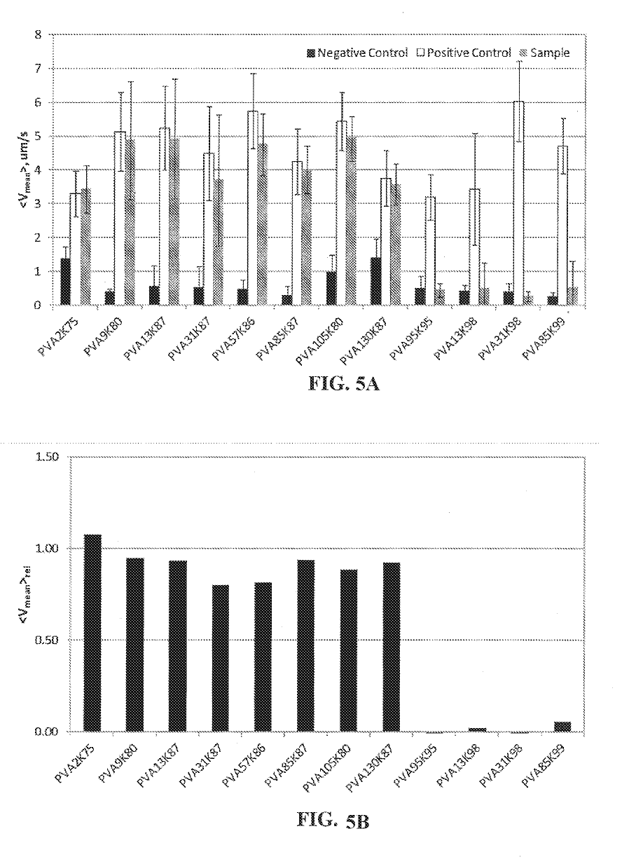

It was discovered that nanoparticles incubated in the presence of certain PVA transported through CVM at the same rate or nearly the same velocity as the positive control. Specifically, the particles stabilized with PVA2K75, PVA9K80, PVA13K87, PVA31K87, PVA57K86, PVA85K87, PVA105K80, and PVA130K87 exhibited <Vₘₑₐₙ> that significantly exceeded those of the negative controls and were indistinguishable, within experimental error, from those of the positive controls. The results are shown in Table 6 and FIG. 5A. For these samples, <Vₘₑₐₙ>ᵣₑₗ values exceeded 0.5, as shown in FIG. 5B.

On the other hand, nanoparticles incubated with PVA95K95, PVA13K98, PVA31K98, and PVA85K99 were predominantly or completely immobilized as demonstrated by respective <Vₘₑₐₙ>ᵣₑₗ values of no greater than 0.1 (Table 6 and FIG. 5B).

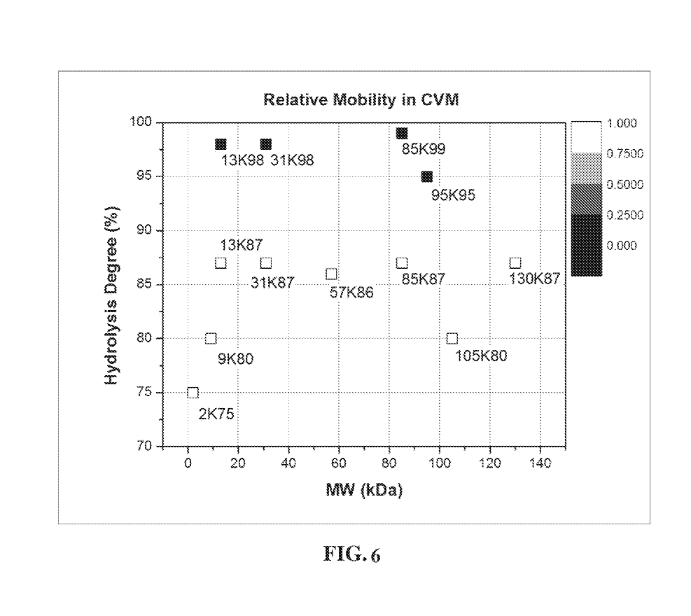

To identify the characteristics of the PVA that render particles mucus penetrating, <Vₘₑₐₙ>ᵣₑₗ of the nanoparticles prepared by incubation with the various PVAs was mapped with respect to MW and hydrolysis degree of the PVAs used (FIG. 6). It was concluded that at least those PVAs that have the hydrolysis degree of less than 95% rendered the nanoparticles mucus-penetrating.

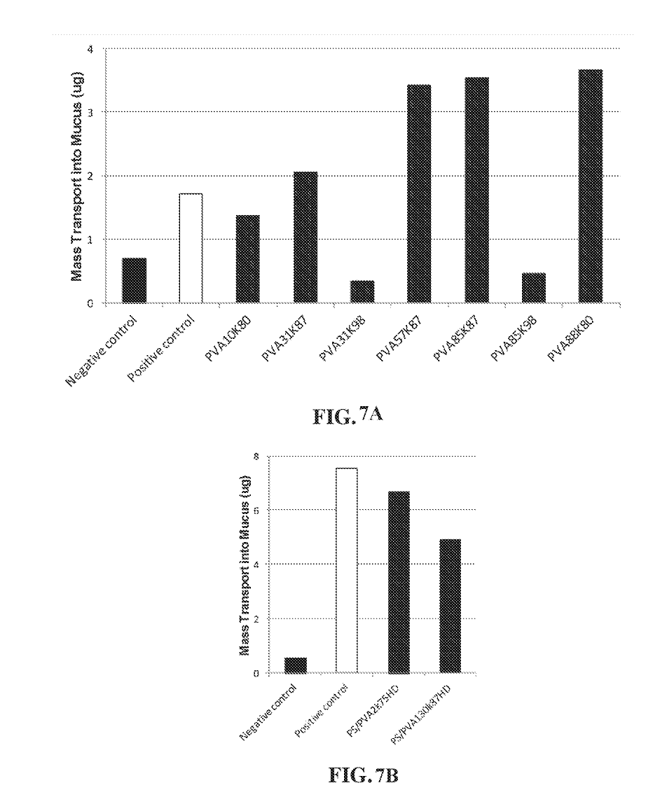

To further confirm the ability of the specific PVA grades to convert mucoadhesive particles into mucus-penetrating particles by physical adsorption, PSCOO⁻ nanoparticles incubated with the various PVAs were tested using the bulk transport assay. In this method, 20 μL of CVM was collected in a capillary tube and one end is sealed with clay. The open end of the capillary tube is then submerged in 20 μL of an aqueous suspension of particles which is 0.5% w/v drug. After the desired time, typically 18 hours, the capillary tube is removed from the suspension and the outside is wiped clean. The capillary containing the mucus sample is placed in an ultracentrifuge tube. Extraction media is added to the tube and incubated for 1 hour while mixing which removes the mucus from the capillary tube and extracts the drug from the mucus. The sample is then spun to remove mucins and other non-soluble components. The amount of drug in the extracted sample can then be quantified using HPLC. The results of these experiments are in good agreement with those of the microscopy method, showing clear differentiation in transport between positive (mucus-penetrating particles) and negative controls (conventional particles). The bulk transport results for PSCOO⁻ nanoparticles incubated with the various PVAs are shown in FIG. 7A-B. These results corroborate microscopy/particle tracking findings with PSCOO⁻ nanoparticles incubated with the various PVAs and demonstrate the incubating nanoparticles with partially hydrolyzed PVAs enhances mucus penetration.

### Example 3

The following describes a non-limiting example of a method of forming mucus-penetrating particles by an emulsification process in the presence of certain poly(vinyl alcohol) polymers (PVA). Polylactide (PLA), a biodegradable pharmaceutically relevant polymer was used as a material to form the core particle via an oil-in-water emulsification process. The PVAs acted as emulsion surface-altering agents and surface-altering agents forming coatings around the produced core particles. PVA of various molecular weights (MW) and hydrolysis degrees were evaluated to determine effectiveness of the formed particles in penetrating mucus.

PLA solution in dichloromethane was emulsified in aqueous solution in the presence of various PVA to determine whether certain PVAs can physically (non-covalently) coat the surface of generated nanoparticles with a coating that would lead to rapid particle penetration in mucus. In these experiments, the PVA acted as a surfactant that forms a stabilizing coating around droplets of emulsified organic phase that, upon solidification, form the core particles. The resulting particles were tested for their mobility in mucus, although in other embodiments, PVA may be exchanged with other surface-altering agents that can increase mobility of the particles in mucus. The PVAs tested ranged in the average molecular weight from 2 kDa to 130 kDa and in the average hydrolysis degree from 75% to 99+%. The PVAs that were tested are listed in Table 2, shown above.

The emulsification-solvent evaporation process was as follows: Approximately 0.5 mL of 20-40 mg/ml solution of PLA (Polylactide grade 100DL7A, purchased from Surmodics) in dichloromethane was emulsified in approximately 4 mL of an aqueous PVA solution (0.5-2 wt %) by sonication to obtain a stable emulsion with the target number-average particle size of <500 nm. Obtained emulsions were immediately subjected to exhaustive rotary evaporation under reduced pressure at room temperature to remove the organic solvent. Obtained suspensions were filtered through 1 micron glass fiber filters to remove any agglomerates. Table 7 lists the particle size characteristics of the nanosuspensions obtained by this emulsification procedure with the various PVA. In all cases, a fluorescent organic dye Nile Red was added to the emulsified organic phase to fluorescently label the resulting particles.

TABLE 7
Particle size measured by DLS in nanosuspensions obtained
by the emulsification process of PLA particles with various PVA.

| PVA Grade | Z-Ave D (nm) | N-Ave D (nm) |
|---|---|---|
| PVA2K75 | 186 | 156 |
| PVA10K80 | 208 | 173 |
| PVA13K98 | 245 | 205 |
| PVA31K87 | 266 | 214 |
| PVA31K98 | 245 | 228 |
| PVA85K87 | 356 | 301 |
| PVA85K99 | 446 | 277 |
| PVA95K95 | 354 | 301 |
| PVA105K80 | 361 | 300 |
| PVA130K87 | 293 | 243 |

The mobility and distribution of the produced nanoparticles in CVM were characterized using fluorescence microscopy and multiple-particle tracking software in a manner similar to that described above. Multiple particle tracking confirmed that in all tested CVM samples the negative controls were constrained, while the positive controls were mobile as demonstrated by the differences in <Vₘₑₐₙ> for the positive and negative controls (Table 8).

TABLE 8
Transport of PLA nanoparticles obtained by the emulsification process with various
PVAs (sample) and controls in CVM: Ensemble-average velocity <Vₘₑₐₙ> (um/s)
and relative sample velocity <Vₘₑₐₙ>ᵣₑₗ.

| | Negative | | Positive | | | | Sample | |
|---|---|---|---|---|---|---|---|---|
| | Control | | Control | | Sample | | (relative) | |
| Stabilizer | <Vₘₑₐₙ> | SD | <Vₘₑₐₙ> | SD | <Vₘₑₐₙ> | SD | <Vₘₑₐₙ>ᵣₑₗ | SD |
| PVA2K75 | 0.95 | 0.64 | 5.5 | 0.92 | 5.51 | 1.2 | 1.00 | 0.39 |
| PVA9K80 | 0.72 | 0.47 | 5.61 | 0.79 | 4.6 | 1.5 | 0.79 | 0.35 |
| PVA31K87 | 0.63 | 0.60 | 4.94 | 1.50 | 3.36 | 1.84 | 0.63 | 0.51 |
| PVA85K87 | 0.57 | 0.4 | 4.49 | 1.21 | 2.9 | 1.56 | 0.59 | 0.45 |
| PVA105K80 | 0.69 | 0.56 | 4.85 | 1.54 | 3.55 | 1.26 | 0.69 | 0.43 |
| PVA130K87 | 0.95 | 0.54 | 4.98 | 1.25 | 3.46 | 1.23 | 0.62 | 0.39 |
| PVA95K95 | 1.39 | 1.28 | 5.72 | 1.57 | 1.63 | 1.5 | 0.06 | 0.46 |
| PVA13K98 | 1.02 | 0.49 | 5.09 | 0.99 | 2.61 | 1.54 | 0.39 | 0.41 |
| PVA31K98 | 1.09 | 0.6 | 5.09 | 0.9 | 2.6 | 1.13 | 0.38 | 0.34 |
| PVA85K99 | 0.47 | 0.33 | 5.04 | 2.2 | 0.81 | 0.77 | 0.07 | 0.19 |

It was discovered that nanoparticles prepared in the presence of certain PVA transported through CVM at the same rate or nearly the same velocity as the positive control. Specifically, the particles stabilized with PVA2K75, PVA9K80, PVA13K87, PVA31K87, PVA85K87, PVA105K80, and PVA130K87 exhibited <Vₘₑₐₙ> that significantly exceeded those of the negative controls and were indistinguishable, within experimental error, from those of the positive controls, as shown in Table 8 and FIG. 8A. For these samples, <Vₘₑₐₙ>ᵣₑₗ values exceeded 0.5, as shown in FIG. 8B.

On the other hand, nanoparticles obtained with PVA95K95, PVA13K98, PVA31K98, and PVA85K99 were predominantly or completely immobilized as demonstrated by respective <Vₘₑₐₙ>ᵣₑₗ values of no greater than 0.4 (Table 8 and FIG. 8B). To identify the characteristics of the PVA that render particles mucus penetrating, <Vₘₑₐₙ>ᵣₑₗ of the nanoparticles prepared with the various PVAs was mapped with respect to MW and hydrolysis degree of the PVAs used (Table 6 and FIG. 8B). It was concluded that at least those PVAs that have the hydrolysis degree of less than 95% rendered the nanoparticles mucus-penetrating.

### Example 4

The following describes a non-limiting example of a method of forming mucus-penetrating non-polymeric solid particles by milling in the presence of certain poly(vinyl alcohol) polymers (PVA). Pyrene, a model hydrophobic compound, was used as the core particle processed by a milling. The PVA acted as milling aids facilitating particle size reduction of the core particles and surface-altering agents forming coatings around the core particles. PVA of various molecular weights (MW) and hydrolysis degrees were evaluated to determine effectiveness of the milled particles in penetrating mucus.

Pyrene was milled in aqueous dispersions in the presence of various PVA to determine whether PVAs of certain MW and hydrolysis degree can: 1) aid particle size reduction to several hundreds of nanometers and 2) physically (non-covalently) coat the surface of generated nanoparticles with a coating that would minimize particle interactions with mucus constituents and prevent mucus adhesion. In these experiments, the PVA acted as a coating around the core particles, and the resulting particles were tested for their mobility in mucus. The PVAs tested ranged in the average molecular weight from 2 kDa to 130 kDa and in the average hydrolysis degree from 75% to 99+%. The PVAs that were tested are listed in Table 1, shown above. A variety of other polymers, oligomers, and small molecules listed in Table 9, including pharmaceutically relevant excipients such as polyvinylpyrrolidones (Kollidon), hydroxypropyl methylcellulose (Methocel), Tween, Span, etc., were tested in a similar manner.

TABLE 9
Other surface-altering agents tested with pyrene as a model compound.

| Chemical Family | Grades |
|---|---|
| Polyvinylpyrrolidone (PVP) | Kollidon 17 |
| | Kollidon 25 |
| | Kollindon 30 |
| PVA-poly(ethylene glycol) graft-copolymer | Kollicoat IR |
| Hydroxypropyl methylcellulose (HPMC) | Methocel E50 |
| | Methocel K100 |
| Non-ionic polyoxyethylene surfactants | Solutol HS 15 |
| | Span 20 |
| | Span 80 |
| | Triton X100 |
| | Tween 20 |
| | Tween 80 |
| | Tyloxapol |
| Non-ionic small molecule surfactants | Octyl glucoside |
| Ionic small molecule surfactants | Cetytrimethylammonium bromide (CTAB) |
| | Sodium dodecyl sulfate (SDS) |

An aqueous dispersion containing pyrene and one of the surface-altering agents listed above was stirred with milling media until particle size was reduced below 500 nm (as measured by dynamic light scattering).

Table 10 lists particle size characteristics of pyrene particles obtained by milling in the presence of the various surface-altering agents. When Span 20, Span 80, or Octyl glucoside was used as surface-altering agents, stable nanosuspensions could not be obtained. Therefore, these surface-altering agents were excluded from further investigation due to their inability to effectively aid particle size reduction.

TABLE 10
Particle size measured by DLS in nanosuspensions obtained
by milling of pyrene with various surface-altering agents.

| Stabilizer | Z-Ave D (nm) | N-Ave D (nm) |
|---|---|---|
| PVA2K75 | 340 | 301 |
| PVA9K80 | 380 | 337 |
| PVA13K87 | 375 | 326 |
| PVA13K98 | 396 | 314 |
| PVA31K87 | 430 | 373 |
| PVA31K98 | 344 | 220 |
| PVA85K87 | 543 | 434 |
| PVA85K99 | 381 | 236 |
| PVA95K95 | 534 | 392 |
| PVA130K87 | 496 | 450 |
| Kollidon 17 | 237 | 163 |
| Kollidon 25 | 307 | 210 |
| Kollindon 30 | 255 | 185 |
| Kollicoat IR | 364 | 192 |
| Methocel E50 | 244 | 160 |
| Methocel K100 | 375 | 216 |
| Tween 20 | 567 | 381 |
| Tween 80 | 553 | 322 |
| Solutol HS | 576 | 378 |
| Triton X100 | 410 | 305 |
| Tyloxapol | 334 | 234 |
| Cremophor RH40 | 404 | 373 |
| Span 20 | not measurable* | |
| Span 80 | not measurable* | |
| Octyl glucoside | not measurable* | |
| SDS | 603 | 377 |
| CTAB | 432 | 354 |

*milling with Span 20, Span 80, Octyl glucoside failed to effectively reduce pyrene particlesize and produce stable nanosuspensions.

The mobility and distribution of the produced pyrene nanoparticles in CVM were characterized using fluorescence microscopy and multiple particle tracking software in a manner similar to that described above. Multiple particle tracking confirmed that in all tested CVM samples the negative controls were constrained, while the positive controls were mobile as demonstrated by the differences in <Vₘₑₐₙ> for the positive and negative controls (Table 11).

TABLE 11
Transport of pyrene nanoparticles (sample) obtained with various surface-altering
agents and controls in CVM: Ensemble-average velocity <Vₘₑₐₙ> (um/s) and relative sample
velocity <Vₘₑₐₙ>ᵣₑₗ.

| | Negative | | Positive | | | | Sample | |
|---|---|---|---|---|---|---|---|---|
| | Control | | Control | | Sample | | (relative) | |
| Stabilizer | <Vₘₑₐₙ> | SD | <Vₘₑₐₙ> | SD | <Vₘₑₐₙ> | SD | <Vₘₑₐₙ>ᵣₑₗ | SD |
| PVA2K75 | 0.4 | 0.24 | 5.73 | 0.73 | 4.73 | 1.08 | 0.81 | 0.24 |
| PVA9K80 | 0.36 | 0.20 | 6.00 | 0.70 | 6.19 | 1.13 | 1.03 | 0.24 |
| PVA13K87 | 1.01 | 1.21 | 5.09 | 0.98 | 4.54 | 1.03 | 0.87 | 0.51 |
| PVA31K87 | 1.28 | 1.14 | 4.88 | 0.6 | 4.57 | 1.123 | 0.91 | 0.55 |
| PVA85K87 | 1.05 | 0.9 | 4.1 | 0.57 | 3.3 | 0.98 | 0.74 | 0.51 |
| PVA130K87 | 0.51 | 0.82 | 5.29 | 0.73 | 4.12 | 1.49 | 0.76 | 0.40 |
| PVA95K95 | 0.4 | 0.27 | 4.53 | 1.03 | 0.67 | 0.6 | 0.07 | 0.16 |
| PVA13K98 | 0.61 | 0.42 | 2.13 | 0.99 | 1.29 | 0.57 | 0.45 | 0.56 |
| PVA31K98 | 0.68 | 0.87 | 5.77 | 1.24 | 2.69 | 2.02 | 0.39 | 0.45 |
| PVA85K99 | 0.43 | 0.23 | 5.42 | 0.97 | 2.23 | 1.60 | 0.36 | 0.33 |
| Kollicoat IR | 0.62 | 0.62 | 5.39 | 0.55 | 0.92 | 0.81 | 0.06 | 0.22 |
| Kollidon 17 | 1.69 | 1.8 | 5.43 | 0.98 | 0.82 | 0.59 | −0.23 | −0.52 |
| Kollidon 25 | 0.41 | 0.34 | 5.04 | 0.64 | 1.29 | 1.09 | 0.19 | 0.25 |
| Kollindon 30 | 0.4 | 0.2 | 4.28 | 0.57 | 0.35 | 0.11 | −0.01 | 0.06 |
| Methocel E50* | | | | | | | | |
| Methocel K100* | | | | | | | | |
| Tween 20 | 0.77 | 0.93 | 5.35 | 1.76 | 1.58 | 2.02 | 0.18 | 0.49 |
| Tween 80 | 0.46 | 0.34 | 3.35 | 1.89 | 0.94 | 0.5 | 0.17 | 0.24 |
| Solutol HS | 0.42 | 0.13 | 3.49 | 0.5 | 0.8 | 0.6 | 0.12 | 0.20 |
| Triton X100 | 0.26 | 0.13 | 4.06 | 1.11 | 0.61 | 0.19 | 0.09 | 0.07 |
| Tyloxapol | 0.5 | 0.5 | 3.94 | 0.58 | 0.42 | 0.23 | −0.02 | −0.16 |
| Cremophor RH40 | 0.48 | 0.21 | 3.2 | 0.97 | 0.49 | 0.24 | 0.00 | 0.12 |
| SDS | 0.3 | 0.12 | 5.99 | 0.84 | 0.34 | 0.15 | 0.01 | 0.03 |
| CTAB | 0.39 | 0.09 | 4.75 | 1.79 | 0.32 | 0.31 | −0.02 | −0.07 |

*Aggregated in CVM, hence not mucus-penetrating (velocity in CVM not measured)

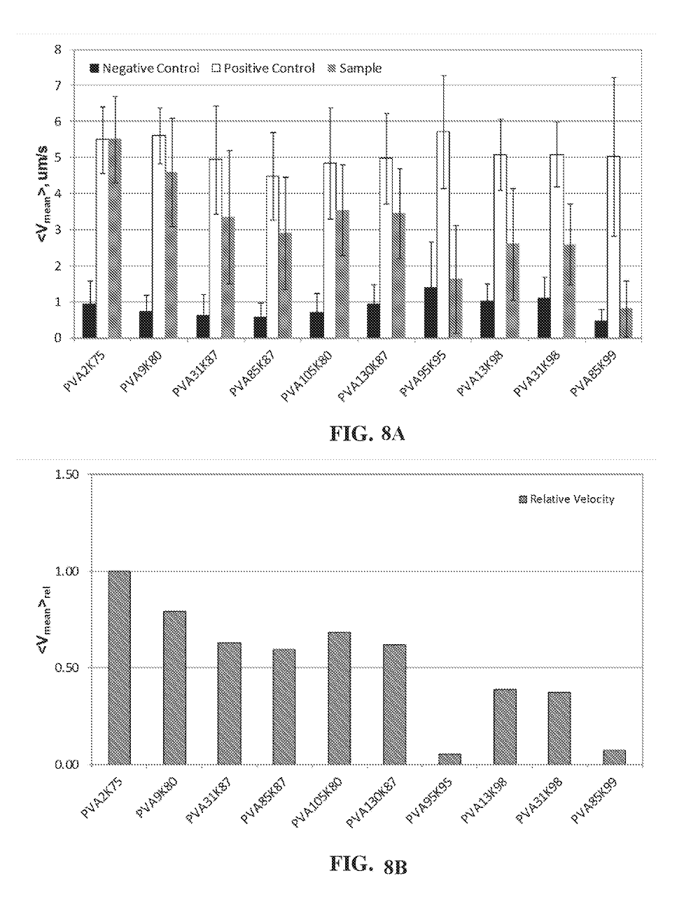

It was discovered that nanoparticles obtained in the presence of certain excipients transported through CVM at the same rate or nearly the same velocity as the positive control. Specifically, pyrene nanoparticles stabilized with PVA2K75, PVA9K80, PVA13K87, PVA31K87, PVA85K87, and PVA130K87 exhibited <Vₘₑₐₙ> that significantly exceeded those of the negative controls and were indistinguishable, within experimental error, from those of the positive controls, as shown in Table 11 and FIG. 9A. For these samples, <Vₘₑₐₙ>ᵣₑₗ values exceeded 0.5, as shown in FIG. 9B.

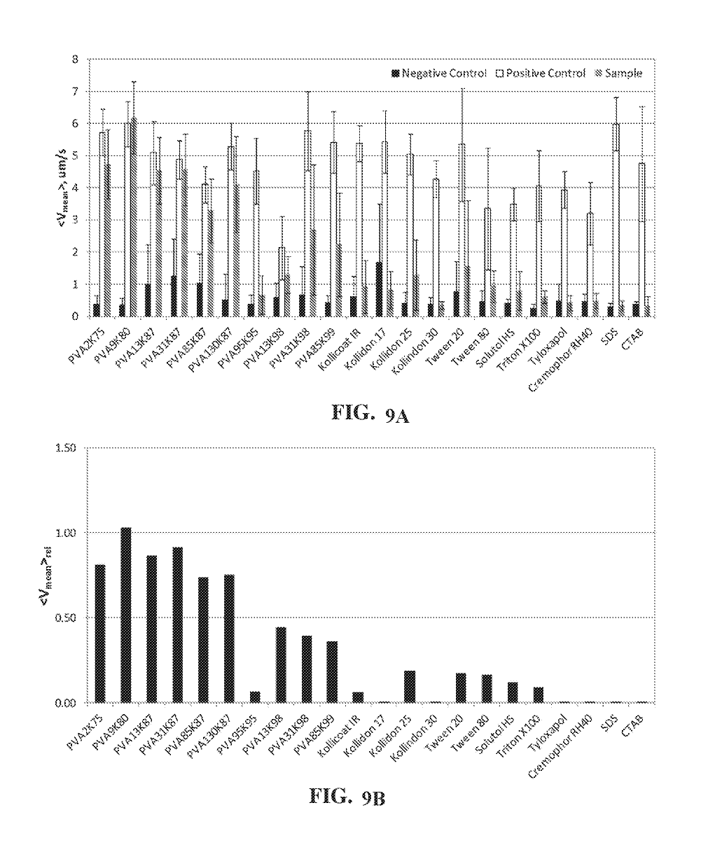

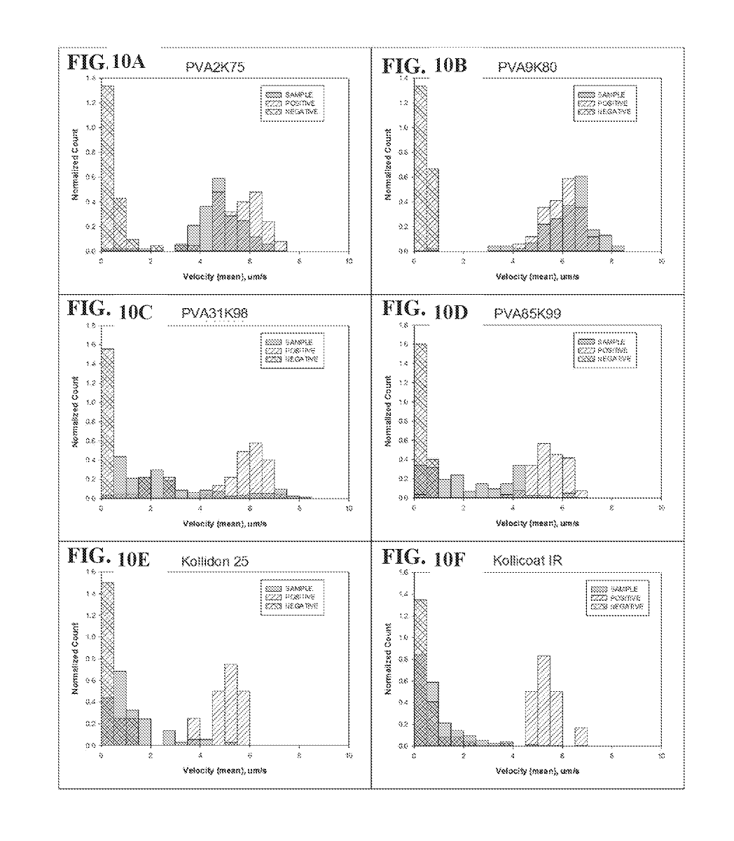

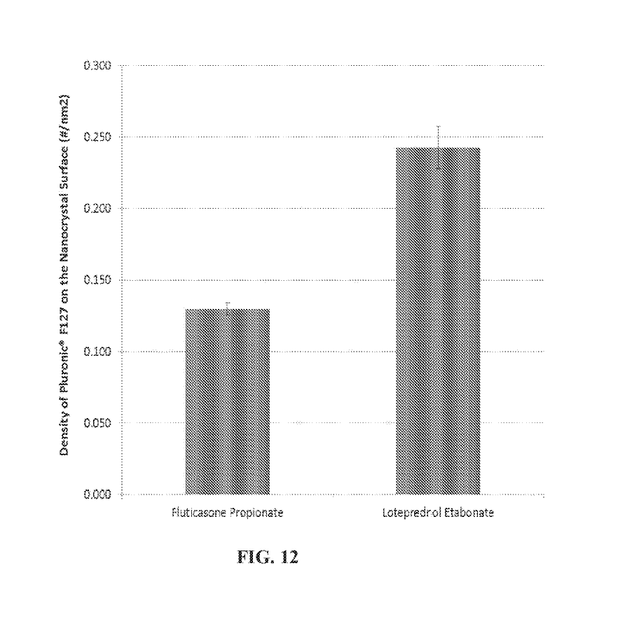

On the other hand, pyrene nanoparticles obtained with the other surface-altering agents, including PVA95K95, PVA13K98, PVA31K98, and PVA85K99, were predominantly or completely immobilized as demonstrated by respective <Vₘₑₐₙ>ᵣₑₗ values of no greater than 0.5 and, with most surface-altering agents, no greater than 0.4 (Table 11 and FIG. 12B). Additionally, FIGS. 10A-10F are histograms showing distribution of Vₘₑₐₙ within an ensemble of particles. These histograms illustrate muco-diffusive behavior of samples stabilized with PVA2K75 and PVA9K80 (similar histograms were obtained for samples stabilized with PVA13K87, PVA31K87, PVA85K87, and PVA130K87, but are not shown here) as opposed to muco-adhesive behavior of samples stabilized with PVA31K98, PVA85K99, Kollidon 25, and Kollicoat IR (chosen as representative muco-adhesive samples).

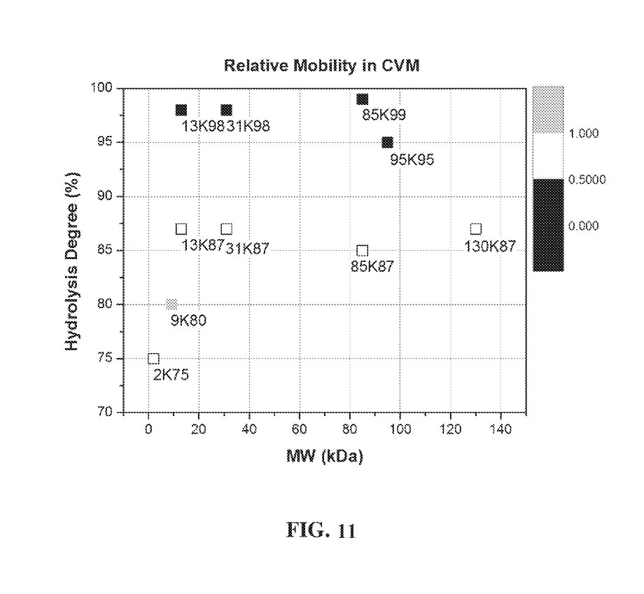

To identify the characteristics of the PVA that render pyrene nanoparticles mucus penetrating, <Vₘₑₐₙ>ᵣₑₗ of the pyrene nanoparticles stabilized with various PVAs was mapped with respect to MW and hydrolysis degree of the PVAs used (FIG. 11). It was concluded that at least those PVAs that have the hydrolysis degree of less than 95% rendered the nanoparticles mucus-penetrating.

### Example 5

This example describes the measurement of the density of Pluronic® F127 on the surface of particles comprising a nanoparticle core of a pharmaceutical agent.

An aqueous dispersion containing a pharmaceutical agent and Pluronic® F127 was milled with milling media until particle size was reduced below 300 nm. A small volume from the milled suspension was diluted to an appropriate concentration (˜100 μg/mL, for example) and the z-average diameter was taken as a representative measurement of particle size. The remaining suspension was then divided into two aliquots. Using HPLC, the first aliquot was assayed for the total concentration of drug (here, loteprednol eltabonate or fluticasone propionate) and for the total concentration of surface-altering moiety (here, Pluronic® F127). Again using HPLC the second aliquot was assayed for the concentration of free or unbound surface-altering moiety. In order to get only the free or unbound surface-altering moiety from the second aliquot, the particles, and therefore any bound surface-altering moiety, were removed by ultracentrifugation. By subtracting the concentration of the unbound surface-altering moiety from the total concentration of surface-altering moiety, the concentration of bound surface-altering moiety was determined. Since the total concentration of drug was also determined from the first aliquot, the mass ratio between the core material and the surface-altering moiety can be determined. Using the molecular weight of the surface-altering moiety, the number of surface-altering moiety molecules to mass of core material can be calculated. To turn this number into a surface density measurement, the surface area per mass of core material needs to be calculated. The volume of the particle is approximated as that of a sphere with the diameter obtained from DLS allowing for the calculation of the surface area per mass of core material. In this way the number of surface-altering moieties per surface area is determined. FIG. 12 shows the results of surface-moiety density determination for loteprednol etabonate and fluticasone propionate.

### Example 6. Formation of Mucus-Penetrating Particles Using Non-Polymeric Solid Particles

The technique described in Example 1 was applied to other non-polymeric solid particles to show the versatility of the approach. F127 was used as the surface-altering agent for coating a variety of active pharmaceuticals used as core particles. Sodium dodecyl sulfate (SDS) was chosen as a negative control so that each drug was compared to a similarly sized nanoparticle of the same compound. An aqueous dispersion containing the pharmaceutical agent and Pluronic® F127 or SDS was milled with milling media until particle size was reduced below 300 nm. Table 12 lists the particle sizes for a representative selection of drugs that were milled using this method.

TABLE 12
Particle sizes for a representative selection of drugs
milled in the presence of SDS and F127.

| | | Z-Ave D | |
|---|---|---|---|
| Drug | Stabilizer | (nm) | PDI |
| Fluticasone | F127 | 203 | 0.114 |
| propionate | SDS | 202 | 0.193 |
| Furosemide | F127 | 217 | 0.119 |
| | SDS | 200 | 0.146 |
| Itraconazole | F127 | 155 | 0.158 |
| | SDS | 168 | 0.163 |
| Prednisolone | F127 | 273 | 0.090 |
| | SDS | 245 | 0.120 |
| Loteprednol | F127 | 241 | 0.123 |
| etabonate | SDS | 241 | 0.130 |
| Budesonide | F127 | 173 | 0.112 |
| | SDS | 194 | 0.135 |
| Indomethacin | F127 | 225 | 0.123 |
| | SDS | 216 | 0.154 |

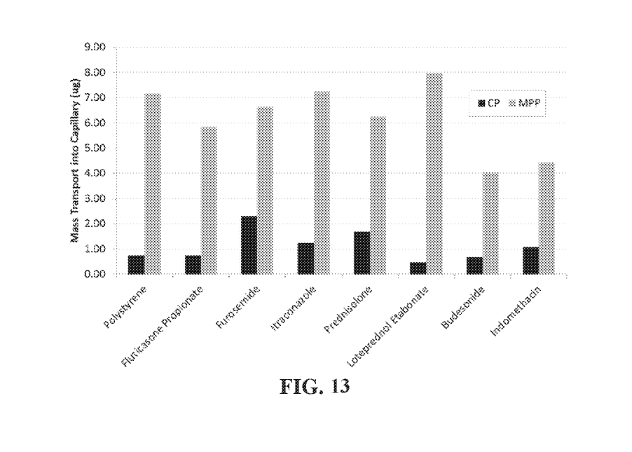

In order to measure the ability of drug nanoparticles to penetrate mucus a new assay was developed which measures the mass transport of nanoparticles into a mucus sample. Most drugs are not naturally fluorescent and are therefore difficult to measure with particle tracking microscopy techniques. The newly-developed bulk transport assay does not require the analyzed particles to be fluorescent or labeled with dye. In this method, 20 μL of CVM is collected in a capillary tube and one end is sealed with clay. The open end of the capillary tube is then submerged in 20 μL of an aqueous suspension of particles which is 0.5% w/v drug. After the desired time, typically 18 hours, the capillary tube is removed from the suspension and the outside is wiped clean. The capillary containing the mucus sample is placed in an ultracentrifuge tube. Extraction media is added to the tube and incubated for 1 hour while mixing which removes the mucus from the capillary tube and extracts the drug from the mucus. The sample is then spun to remove mucins and other non-soluble components. The amount of drug in the extracted sample can then be quantified using HPLC. The results of these experiments are in good agreement with those of the microscopy method, showing clear differentiation in transport between mucus penetrating particles and conventional particles. The transport results for a representative selection of drugs are shown in FIG. 13. These results corroborate microscopy/particle tracking findings with pyrene and demonstrate the extension to common active pharmaceutical compounds; coating non-polymeric solid nanoparticles with F127 enhances mucus penetration.

### Example 7. Synthesis of Hydrocortisone Derivatives

All compounds were synthesized as described below. The LC-MS method that supported the synthesis is as follows: column—Waters XTerra® MS C18, 3.5 μm, 3.0×150 mm, column temperature—40° C., flow rate—0.6 mL/min, detection wavelength—254 nm, flow gradient—98:2 (0 minutes) to 0:100 (10 minutes) 0.1% formic acid/H₂O:0.1% formic acid/acetonitrile.

### Compound 2: 17-[(2-Furanylcarbonyl)oxy]-11,21-dihydroxy-(11β)-pregn-4-ene-3,20-dione

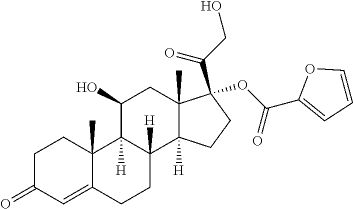

### Synthesis of 2-furancarboximidic acid methyl ester hydrochloride

Methanol (125 mL) was cooled in ice bath. Acetyl chloride (66.0 mL, 0.92 mol) was slowly added. The solution was stirred while cooling in the ice bath for 1 hour. The solution was stirred further for 1 hour at room temperature. Furan-2-carbonitrile (75.0 g, 0.81 mol) was added and the reaction was stirred overnight. Diethyl ether (500 mL) was added and then suspension was stirred for 30 minutes. The precipitate was filtered. The solid was washed with additional diethyl ether (100 mL) to obtain product as an off-white solid. Yield 74.0 g, 56%.

### Synthesis of 2-(trimethoxymethyl) furan

Potassium carbonate (300 g) was dissolved in water (1 L). The solution was cooled to room temperature and 2-furancarboximidic acid methyl ester hydrochloride (100.0 g, 0.62 mol) was added with stirring. The mixture was extracted with ethyl acetate (2×500 mL). The organic solution was dried with anhydrous magnesium sulfate and the solvent was evaporated. The residue was treated with hexanes (500 mL) and the solids were filtered. The solvent was evaporated leaving colorless oil (62.0 g). The oil was added to a solution of phosphoric acid (anhydrous, 48.2 g, 0.49 mol) in dry methanol (700 mL). The solution was refluxed for 6 hours, then the precipitate was filtered and the solvent was evaporated. Hexanes (500 mL) was added to the residue and the precipitate was filtered. The solvent was evaporated to obtain the product as colorless oil. Yield 55.0 g, 52%.

### Synthesis of 17-[(2-furanylcarbonyl)oxy]-11,21-dihydroxy-(11β)-pregn-4-ene-3,20-dione (Compound 1)

Hydrocortisone (25.00 g, 69.0 mmol), 2-(trimethoxymethyl)-furan (55.0 g, 320.0 mmol) and pyridinium p-toluenesulfonate (6.5 g, 25.9 mmol) were dissolved in tetrahydrofuran (250 mL). The solution was heated to 70° C. for 3 hours. The solvent was evaporated. Dichloromethane (400 mL) was added followed by addition of hydrochloric acid (1.0 M, 200 mL). The mixture was vigorously stirred for 30 minutes. The organic phase was separated and dried with anhydrous magnesium sulfate. The solvent was evaporated and the residue was dissolved in hexanes:dichloromethane 9:1 (500 mL). The solution was applied on silica pad (300 g). The impurities were eluted with hexanes and the product mixture was eluted with dichloromethane:ethyl acetate 3:7. The solvent was evaporated leaving a white solid (28.0 g) that consisted mostly of two isomers. The major isomer was separated by flash chromatography (330 g silica column, hexanes to ethyl acetate). The fractions containing major isomer were combined and concentrated to ca. 100 mL. The solution was sonicated to induce formation of solid product, then the product was filtered and dried in high vacuum overnight to obtain a white solid. Yield: 13.0 g, 41%. LC-MS: retention time 8.33 minutes, MS (positive ion) 345.2 (50%), 457.3 (100%, M+1), 458.3 (30%, M+2), MS (negative ion) 491.1 (20%), 501.2 (100%). ¹H NMR (CDCl₃) −7.61 (dd, J=2.0, 1.0 Hz, 1H), 7.19 (dd, J=3.5, 1.0 Hz, 1H), 6.53 (dd, J=4.0, 2.0 Hz, 1H), 5.71-5.70 (m, 1H), 4.55-4.54 (m, 1H), 4.36 (dd, J=4.5, 23.0 Hz, 2H), 3.09 (br. s., 1H), 2.95-2.85 (m, 1H), 2.56-2.44 (m, 2H), 2.42-2.34 (m, 1H), 2.30-2.18 (m, 3H), 2.16-1.71 (m, 7H), 1.57-1.46 (m, 1H), 1.46 (m, 3H), 1.27-1.09 (m, 3H), 0.99 (s, 3H).

### Compound 3: 17-[[2-(4-Bromophenyl)acetyl]oxy]-11,21-dihydroxy-(11β)-pregn-4-ene-3,20-dione

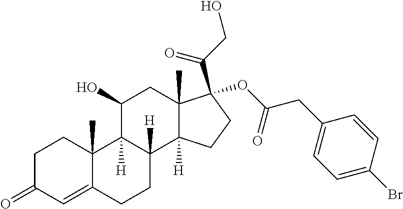

### Synthesis of 4-bromobenzeneethanimidic acid methyl ester hydrochloride

4-Bromobenzeneacetonitrile (50.0 g, 0.26 mol) was dissolved in dry methanol (13 mL). Hexanes (125 mL) was added. The reaction mixture was cooled in ice bath and saturated with hydrogen chloride gas (generated from 100 mL of concentrated hydrochloric acid slowly added to 250 mL of concentrated sulfuric acid). The ice bath was removed and the mixture was stirred overnight. The solution was decanted from the formed semi solid. The semi-solid was suspended in diethyl ether (250 mL). The suspension was stirred for 30 minutes and the solid was filtered. The solid was washed with ether (100 mL). The solid was dried on the funnel by passage of vacuum for 30 minutes to obtain product as a white solid. Yield 69.0 g, 100%.

### Synthesis of 1-bromo-4-(2,2,2-trimethoxyethyl)-benzene

4-Bromobenzeneethanimidic acid methyl ester hydrochloride (69.0 g, 0.26 mol) was dissolved in dry methanol (80 mL). The solution was stirred for 3 days. The precipitate was filtered and rinsed with diethyl ether (100 mL). The solution was evaporated. Diethyl ether (250 mL) was added. The precipitate was filtered and the solvent was evaporated. Trace solvent was removed under high vacuum overnight to obtain product as a colorless oil. Yield 63.7 g, 89%.

### Synthesis of 17-[[2-(4-bromophenyl)acetyl]oxy]-11,21-dihydroxy-(11β)-pregn-4-ene-3,20-dione (Compound 3)

Hydrocortisone (21.00 g, 58.0 mmol), (2,2,2-trimethoxyethyl)benzene (63.0 g, 229.1 mmol) and pyridinium p-toluenesulfonate (5.4 g, 21.5 mmol) were dissolved in tetrahydrofuran (300 mL). The solution was heated to 70° C. for 3 hours. The solvent was evaporated. Diethyl ether (300 mL) was added and the precipitate was filtered, then dissolved in dioxane (500 mL). Hydrochloric acid (1.0 M, 100 mL) was added. The mixture was vigorously stirred for 1 hour. The solution was diluted with water (3.5 L) and stirred for 30 minutes. The precipitate was filtered and dissolved in dichloromethane (300 mL). The solution was dried with anhydrous magnesium sulfate, then solvent was evaporated to leave a residue that that consisted mostly of two isomers. The mixture was dissolved in ethyl acetate (500 mL). The solution was concentrated (ca. 100 mL) and sonicated. The precipitate was filtered leaving behind a white solid (17.6 g), which was further purified by flash chromatography in dichloromethane to dichloromethane:ethyl acetate 1:1 (330 g silica column). The fractions containing the more polar compound were concentrated to ca. 50 mL and hexanes was added until the solution became cloudy. The suspension was sonicated to induce formation of solid product, which were filtered and dried in high vacuum overnight to obtain the product as a white solid. Yield: 12.91 g, 40%. LC-MS: LC retention time 9.22 minutes; MS (positive ion): 559.2 (100%, M+1), 560.2 (30%, M+2), 561.2 (100%, M+1), 562.1 (30%, M+2), MS (negative ion): 539.2 (100%), 540.2 (30%), 541.2 (100%), 542.2 (30%), 593.2 (15%), 595.2 (15%). ¹H NMR (CDCl₃) −7.47-7.43 (m, 2H), 7.12-7.07 (m, 2H), 5.71 (br. s., 1H), 4.42 (br. s., 1H), 4.31-4.14 (2H), 3.58 (s, 2H), 3.03-3.01 (m, 1H), 2.76-2.67 (m, 1H), 2.54-2.34 (m, 3H), 2.29-2.16 (m, 2H), 2.03-1.53 (m, 10H), 1.41 (s, 3H), 1.11-0.95 (2H), 0.90 (s, 3H).

### Compound 1: 11,21-Dihydroxy-17-[1-oxo-3-(phenylsulfonyl)propoxy]-(11β)-pregn-4-ene-3,20-dione

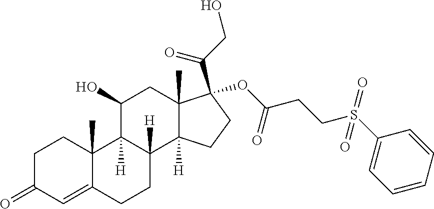

### Synthesis of 3-(phenylthio)propanenitrile

Thiophenol (40 mL, 0.39 mol) was dissolved in methanol (720 mL). Triethylamine was added (2.4 mL, 17.3 mmol). Acrylonitrile (23.2 mL, 0.36 mol) was added dropwise over 1 hour. The solution was stirred overnight. The solvents were evaporated and the residual solvents were removed under high vacuum for 30 minutes at 70° C. to obtain the compound as a yellow oil. Yield 59.4 g, 93%.

### Synthesis of 3-(phenylthio)propanimidic acid ethyl ester hydrochloride

3-(Phenylthio)propanenitrile (59.4 g, 0.36 mol) was dissolved in dichloromethane (300 ml) and ethanol (32 mL). The solution was cooled to −35° C. and saturated with hydrogen chloride gas (generated from 150 mL of concentrated hydrochloric acid slowly added to 300 mL of concentrated sulfuric acid over 1 hour. The cooling bath was removed and the mixture was stirred overnight. The solvents were evaporated. The residual ethanol was co-evaporated with dichloromethane (3×300 mL) to produce product as a thick colorless oil. Yield 97.4 g, 100%, containing ca. 10% residual solvent.

### Synthesis of (3,3,3-triethoxypropyl)(phenyl)sulfane

3-(Phenylthio)propanimidic acid ethyl ester hydrochloride (97.4 g, 0.36 mol) was dissolved in dichloromethane (300 mL) and dry ethanol (200 mL). The reaction mixture was stirred for 3 days. The precipitate was filtered. Diethyl ether (350 mL) was added and the precipitate was filtered. The addition of ether and filtration was repeated. The residual solvent was removed under high vacuum for 2 hours to obtain the product as a yellow oil. Yield 78.0 g, 76%.

### Synthesis of 11,21-Dihydroxy-17-[1-oxo-3-(phenylthio)propoxy]-(11β)-pregn-4-ene-3,20-dione

Hydrocortisone (20.00 g, 55.2 mmol), (3,3,3-triethoxypropyl)(phenyl)sulfane (32.0 g, 112.7 mmol) and pyridinium p-toluenesulfonate (5.2 g, 20.7 mmol) were dissolved in tetrahydrofuran (300 mL). The solution was heated to 70° C. for 3 hours. The solvent was evaporated. Dichloromethane (300 mL) was added followed by addition of hydrochloric acid (1.0 M, 300 mL). The mixture was vigorously stirred for 2 hours. The organic phase was separated, washed with water (300 mL) and dried with anhydrous magnesium sulfate. The solvent was evaporated leaving an oily residue that consisted mostly of two isomers. The material was dissolved in dichloromethane (50 mL) and applied in silica column (330 g), then separated by flash chromatography using dichloromethane to dichloromethane:ethyl acetate 1:1. The fractions containing the more polar isomer were evaporated, leaving the product as a colorless oil. Yield 20.0 g, 69%, containing ca. 30% residual solvent. LC-MS: LC retention time 9.34 minutes, MS (positive ion) 527.3 (100%, M+1), 528.3 (30%), MS (negative ion): 461.2 (40%), 561.2 (80%), 562.2 (25%), 571.3 (100%), 572.3 (30%).

### Synthesis of 11,21-dihydroxy-17-[1-oxo-3-(phenylsulfonyl)propoxy]-(11β)-pregn-4-ene-3,20-dione (Compound 2)

11,21-Dihydroxy-17-[1-oxo-3-(phenylthio)propoxy]-(11β)-pregn-4-ene-3,20-dione (20.0 g, 26.6 mmol) was dissolved in dichloromethane (500 mL). The solution was cooled in ice bath. mCPBA (20.0 g, 89.5 mmol, 77%) was dissolved in dichloromethane (200 mL), then the mCPBA solution was added over 1 hour. The solution was stirred for 1 hour, and then washed with aqueous sodium hydroxide (1.0 M, 2×300 mL) and water (300 mL). The solution was dried with anhydrous magnesium sulfate and the solvent was concentrated to 100 mL. The material was applied on silica column (330 g) and purified by flash chromatography using dichloromethane to ethyl acetate. The solvent was evaporated and dried in high vacuum overnight to obtain product as a white foam. Yield 12.5 g, 84%. LC-MS: LC retention time 8.28 minutes, MS (positive ion) 559.2 (100%, M+1), 560.3 (30%), MS (negative ion) 557.3 (80%), 593.2 (25%), 603.3 (100%). ¹H NMR (CDCl₃) −7.93-7.88 (m, 2H), 7.72-7.24 (m, 1H), 7.62-7.55 (m, 2H), 5.70 (br. s., 1H), 4.49 (br. s., 1H), 4.32 (q, J=18.5 Hz, 2H), 3.36 (td, J=7.5, 1.5 Hz, 2H), 3.02 (br. s., 1H), 2.78 (td, J=7.5, 1.5 Hz, 2H), 2.75-2.68 (m, 1H), 2.55-2.33 (m, 3H), 2.30-2.15 (m, 2H), 2.11-1.98 (m, 2H), 1.95-1.80 (m, 3H), 1.68-0.58 (4H), 1.52-1.44 (m, 1H), 1.44 (s, 3H), 1.22-1.04 (m, 2H), 0.95 (s, 3H).

### Example 8: Formulation of Compounds as Mucus-Penetrating Particles

### Media Milling

All compounds were formulated using excipients and processes that can produce MPPs. Specifically, the compounds were milled in the presence of Pluronic® F127 (F127) to 1) aid particle size reduction to several hundreds of nanometers and 2) physically (non-covalently) coat the surface of generated nanoparticles with a coating that would minimize particle interactions with mucus constituents and prevent mucus adhesion.

A milling procedure was employed in which aqueous dispersions containing coarse compound particles were individually milled with F127 at near-neutral pH buffer using a grinding medium. Briefly, a slurry containing 5% of compound and 5% F127 in PBS (0.0067 M PO₄³⁻), pH 7.1 was added to an equal bulk volume of 1-mm ceria-stabilized zirconium oxide beads in a glass vial (e.g., 2 mL of slurry per 2 mL of beads). A magnetic stir bar was used to agitate the beads, stirring at approximately 500 rpm at ambient conditions for 25 hours.

The milled suspensions were subjected to dynamic light scattering (DLS) measurements to determine particle size and polydispersity index (PDI, a measure of the width of the particle size distribution). The samples for DLS measurements were buffered with HyClone™ PBS (Phosphate-Buffered Saline) to produce isotonic samples that have a physiologically relevant pH.

Table 13 summarizes the particle size and PDI of each compound after milling. The particle size and PDI of the milled suspensions of compounds 1 and 2 were reduced to <350 nm (z-averaged) and <0.20, respectively (Table 1). The purity of both compounds, as determined by high-performance liquid chromatography (HPLC), prior to milling was >96%. After milling, purity remained >96. The purity of Compound 7 after milling was <90%.

TABLE 13
Size (Z-averaged), PDI and chemical purity of milled suspensions.

| Compound | Size | PDI | Purity after milling |
|---|---|---|---|
| 2 | 238 | 0.089 | >96% |
| 3 | 341 | 0.197 | >96% |
| 1 | 509 | 0.318 | <90% |

The HPLC method used to determine the purity of milled suspensions is as follows: column—SunFire™ C18, 3.5 μm, 3.0×150 mm, column temperature—40° C., flow rate—0.7 mL/min, detection wavelength—254 nm, flow gradient—50:50 (0 minutes) to 0:100 (10 minutes) 0.1% phosphoric acid/H₂O:acetonitrile.

### Example 9. Crystalline Forms

### Sample Preparation, Procedure A for Milled Samples.

Particles were isolated by centrifugation, then resuspended in H₂O and then recentrifuged. The wet sample was resuspended in H₂O and deposited thinly and evenly onto a flat zero background sample holder (Rigaku 906165). The sample was allowed to air dry.

### Sample Preparation, Procedure B for Neat Compound Samples.

Milligram amounts were packed as an evenly thin layer of solid onto a zero background sample holder (Rigaku 906165).

### Data Acquisition

XRPD patterns were obtained using a Rigaku MiniFlex 600 benchtop x-ray diffractometer equipped with a Cu X-ray tube (Cu/Kα=1.54059 Å), a six-position sample changer and a D/teX Ultra detector. XRPD patterns were acquired from 3-40° two theta at 0.02° step size and 5°/min scan speed using the following instrument settings: 40 kV-15 mA X-ray generator, 2.5° Soller Slit, 10 mm IHS, 0.625° Divergence Slit, 8 mm Scatter Slit with Kβ filter, and an open Receiving Slit. Diffraction patterns were viewed and analyzed using PDXL analysis software provided by the instrument manufacturer. A reference standard silicon powder (NIST Standard Reference Material 640d) generated a peak at 28.43° and 28.45° two theta when samples were prepared as suspension in H₂O (to simulate Procedure A) and Procedure B, respectively.

### Samples

Except for Crystalline Form 1-B, the XRPD samples of all other forms were prepared using Procedure A (milled compounds) or Procedure B (neat compounds). Procedure A was used to prepare the XRPD sample of Crystalline Form 1-B.

### Results

The crystal form summary of the input (before milling) and milled crystal forms is shown in Table 14. All input crystalline forms were arbitrarily designated as “A” forms. New forms that emerged after milling, if any, were sequentially designated as “B”, “C”, etc. An “A” form was not designated for Compound 2 since the input material was amorphous.

TABLE 14
Summary of Crystal Forms Before and After Milling.

| | Crystal Forms | | Crystal form change |
|---|---|---|---|
| | Input | Final | after milling |
| Compound | (before milling) | (after milling) | of Input forms? |
| 2 | 2-A | 2-A | No |
| 3 | 3-A | 3-B | Yes |
| 1 | Amorphous | 1-B | Yes |

Compound 2 did not change crystal form after milling while compounds 1 and 3 changed forms after milling. Neat Form 3-B was prepared to demonstrate that it can be milled without undergoing further crystal form change. Neat form 1-B was not prepared due to chemical instability of compound during milling. Briefly, an aqueous suspension (approximately 400 mg in 4-6 mL H₂O) of 3-A was stirred at 40° C. for 1 day to produce 3-B. The crystal form conversion experiment is described in Table 15.

TABLE 15
Summary of neat crystal form conversion.

| | Crystal Form | | | |
|---|---|---|---|---|
| | Input | Final | Stirring | Stirring |
| | (before | (after | temperature | time |
| Compound | stirring) | stirring) | (° C.) | (days) |
| 3 | 3-A | 3-B | 40 | 1 |

Form 3-B was wet-milled using the same method that generated the data in Tables 13 and 14. A comparison of milled particle size and PDI between the input form 3-A and 3-B is shown in Table 16. Data shows that crystal form of the input “B” material was preserved during milling.

TABLE 16
Size (Z-average) and PDI of suspensions using different starting forms.

| | Input | Milled Suspension | | |
|---|---|---|---|---|
| Compound | Form* | Final Form | Size (nm) | PDI |
| 3 | A | B | 341 | 0.197 |
| | B | B | 262 | 0.154 |

*Data for input 3-A form were taken from Table 13.

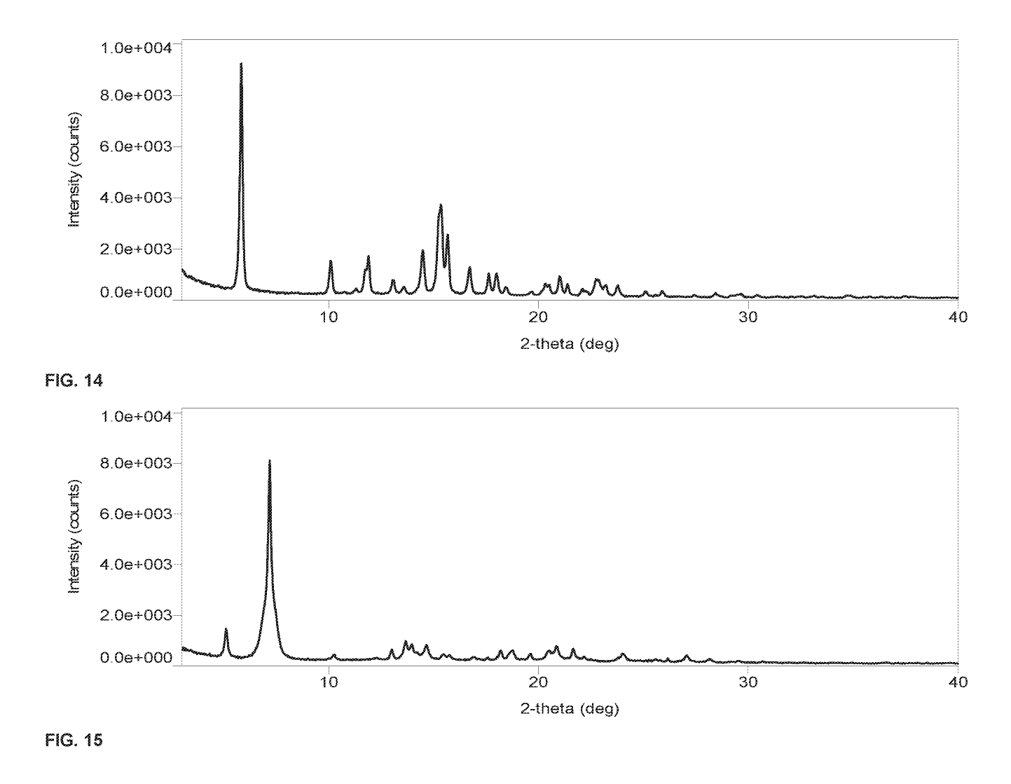

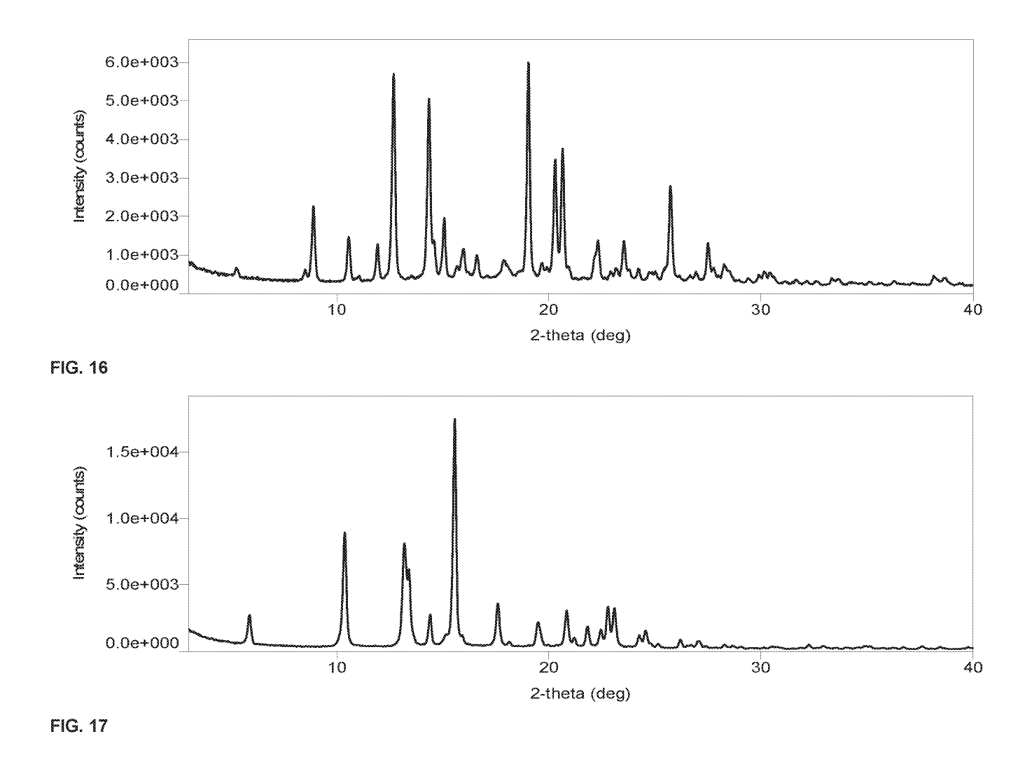

A summary of the XRPD peaks is tabulated in Tables 17-20 and the XRPD patterns of Forms 2-A, 3-A, 3-B and 1-B are shown in FIGS. 14-17.

TABLE 17
XRPD Peak Listing for Crystalline Form 2-A.

| | Position ± 0.2 | d-spacing ± 0.2 | Relative Intensity |
|---|---|---|---|
| No. | [°2θ] | [Å] | [%] |
| 1 | 5.83 | 15.14 | 100.0 |
| 2 | 10.09 | 8.76 | 14.7 |
| 3 | 11.31 | 7.82 | 1.2 |
| 4 | 11.72 | 7.54 | 11.5 |
| 5 | 11.88 | 7.45 | 10.5 |
| 6 | 13.06 | 6.77 | 5.5 |
| 7 | 13.57 | 6.52 | 2.3 |
| 8 | 14.49 | 6.11 | 23.3 |
| 9 | 15.32 | 5.78 | 66.0 |
| 10 | 15.66 | 5.65 | 24.9 |
| 11 | 16.72 | 5.30 | 12.4 |
| 12 | 17.61 | 5.03 | 7.7 |
| 13 | 17.98 | 4.93 | 10.0 |
| 14 | 18.43 | 4.81 | 3.5 |
| 15 | 19.65 | 4.51 | 1.4 |
| 16 | 20.35 | 4.36 | 7.5 |
| 17 | 20.51 | 4.33 | 1.9 |
| 18 | 21.00 | 4.23 | 9.9 |
| 19 | 21.36 | 4.16 | 4.5 |
| 20 | 22.09 | 4.02 | 4.6 |
| 21 | 22.75 | 3.91 | 16.1 |
| 22 | 23.16 | 3.84 | 3.9 |
| 23 | 23.72 | 3.75 | 5.2 |
| 24 | 25.10 | 3.54 | 2.4 |
| 25 | 25.88 | 3.44 | 3.1 |
| 26 | 27.40 | 3.25 | 0.7 |
| 27 | 28.45 | 3.14 | 1.2 |
| 28 | 28.63 | 3.11 | 0.8 |
| 29 | 29.62 | 3.01 | 2.9 |
| 30 | 30.43 | 2.94 | 1.1 |
| 31 | 33.16 | 2.70 | 1.1 |
| 32 | 34.74 | 2.58 | 1.6 |
| 33 | 35.77 | 2.51 | 0.4 |
| 34 | 36.33 | 2.47 | 0.4 |
| 35 | 36.80 | 2.44 | 0.2 |
| 36 | 37.42 | 2.40 | 1.4 |

TABLE 18
XRPD Peak Listing for Crystalline Form 3-A.

| | Position ± 0.2 | d-spacing ± 0.2 | Relative Intensity |
|---|---|---|---|
| No. | [°2θ] | [Å] | [%] |
| 1 | 5.08 | 17.37 | 6.6 |
| 2 | 7.18 | 12.31 | 100.0 |
| 3 | 10.25 | 8.63 | 1.6 |
| 4 | 12.25 | 7.22 | 3.0 |
| 5 | 12.99 | 6.81 | 2.7 |
| 6 | 13.67 | 6.47 | 2.0 |
| 7 | 13.90 | 6.37 | 14.9 |
| 8 | 14.64 | 6.05 | 5.2 |
| 9 | 15.60 | 5.68 | 5.1 |
| 10 | 16.87 | 5.25 | 4.7 |
| 11 | 18.18 | 4.88 | 3.2 |
| 12 | 18.73 | 4.73 | 6.3 |
| 13 | 19.63 | 4.52 | 2.9 |
| 14 | 20.45 | 4.34 | 9.0 |
| 15 | 20.83 | 4.26 | 5.4 |
| 16 | 21.63 | 4.11 | 3.8 |
| 17 | 22.15 | 4.01 | 4.0 |
| 18 | 24.00 | 3.71 | 5.3 |
| 19 | 25.58 | 3.48 | 2.8 |
| 20 | 26.16 | 3.40 | 0.6 |
| 21 | 27.04 | 3.30 | 3.3 |
| 22 | 28.12 | 3.17 | 1.1 |
| 23 | 29.52 | 3.02 | 0.7 |
| 24 | 36.58 | 2.45 | 0.1 |

TABLE 19
XRPD Peak Listing for Crystalline Form 3-B.

| | Position ± 0.2 | d-spacing ± 0.2 | Relative Intensity |
|---|---|---|---|
| No. | [°2θ] | [Å] | [%] |
| 1 | 5.25 | 16.81 | 4.1 |
| 2 | 8.49 | 10.41 | 4.1 |
| 3 | 8.88 | 9.95 | 36.7 |
| 4 | 10.55 | 8.38 | 19.6 |
| 5 | 11.01 | 8.03 | 1.9 |
| 6 | 11.91 | 7.43 | 13.6 |
| 7 | 12.66 | 6.98 | 100.0 |
| 8 | 14.34 | 6.17 | 80.3 |
| 9 | 14.57 | 6.08 | 17.4 |
| 10 | 15.05 | 5.88 | 24.8 |
| 11 | 15.66 | 5.65 | 4.3 |
| 12 | 15.97 | 5.55 | 15.3 |
| 13 | 16.58 | 5.34 | 9.2 |
| 14 | 17.88 | 4.96 | 14.5 |
| 15 | 19.02 | 4.66 | 97.8 |
| 16 | 19.64 | 4.52 | 10.3 |
| 17 | 20.28 | 4.37 | 59.0 |
| 18 | 20.63 | 4.30 | 59.8 |
| 19 | 22.14 | 4.01 | 9.0 |
| 20 | 22.30 | 3.98 | 13.3 |
| 21 | 22.90 | 3.88 | 2.1 |
| 22 | 23.15 | 3.84 | 4.1 |
| 23 | 23.52 | 3.78 | 16.0 |
| 24 | 23.75 | 3.74 | 4.3 |
| 25 | 24.20 | 3.67 | 2.9 |
| 26 | 24.78 | 3.59 | 3.4 |
| 27 | 25.01 | 3.56 | 1.2 |
| 28 | 25.47 | 3.49 | 6.2 |
| 29 | 25.71 | 3.46 | 37.9 |
| 30 | 26.65 | 3.34 | 0.6 |
| 31 | 26.92 | 3.31 | 1.9 |
| 32 | 27.48 | 3.24 | 14.1 |
| 33 | 27.74 | 3.21 | 5.4 |
| 34 | 28.30 | 3.15 | 13.2 |
| 35 | 29.36 | 3.04 | 1.0 |
| 36 | 29.87 | 2.99 | 3.2 |
| 37 | 30.12 | 2.96 | 4.5 |
| 38 | 30.40 | 2.94 | 6.4 |
| 39 | 31.08 | 2.87 | 0.7 |
| 40 | 31.62 | 2.83 | 1.5 |
| 41 | 32.14 | 2.78 | 1.0 |
| 42 | 32.61 | 2.74 | 1.1 |
| 43 | 33.34 | 2.69 | 1.9 |
| 44 | 33.62 | 2.66 | 3.0 |
| 45 | 35.09 | 2.56 | 0.9 |
| 46 | 35.59 | 2.52 | 0.4 |
| 47 | 36.26 | 2.48 | 1.6 |
| 48 | 37.11 | 2.42 | 0.6 |
| 49 | 38.14 | 2.36 | 6.7 |
| 50 | 38.61 | 2.33 | 4.6 |

TABLE 20
XRPD Peak Listing for Crystalline Form 1-B.

| | Position ± 0.2 | d-spacing ± 0.2 | Relative Intensity |
|---|---|---|---|
| No. | [°2θ] | [Å] | [%] |
| 1 | 5.88 | 15.03 | 12.2 |
| 2 | 10.36 | 8.53 | 57.1 |
| 3 | 13.18 | 6.71 | 53.4 |
| 4 | 13.40 | 6.60 | 28.8 |
| 5 | 14.40 | 6.15 | 10.7 |
| 6 | 15.12 | 5.86 | 2.5 |
| 7 | 15.55 | 5.69 | 100.0 |
| 8 | 15.91 | 5.57 | 2.4 |
| 9 | 17.57 | 5.04 | 20.8 |
| 10 | 18.12 | 4.89 | 1.0 |
| 11 | 19.47 | 4.56 | 11.7 |
| 12 | 19.94 | 4.45 | 0.2 |
| 13 | 20.82 | 4.26 | 16.1 |
| 14 | 21.18 | 4.19 | 2.9 |
| 15 | 21.81 | 4.07 | 7.2 |
| 16 | 22.43 | 3.96 | 6.6 |
| 17 | 22.77 | 3.90 | 16.6 |
| 18 | 23.07 | 3.85 | 17.5 |
| 19 | 24.26 | 3.67 | 5.9 |
| 20 | 24.55 | 3.62 | 7.1 |
| 21 | 25.13 | 3.54 | 0.8 |
| 22 | 26.19 | 3.40 | 2.7 |
| 23 | 26.65 | 3.34 | 1.8 |
| 24 | 27.07 | 3.29 | 3.5 |
| 25 | 27.39 | 3.25 | 0.6 |
| 26 | 27.83 | 3.20 | 0.3 |
| 27 | 28.28 | 3.15 | 1.4 |
| 28 | 28.67 | 3.11 | 1.3 |
| 29 | 29.05 | 3.07 | 0.5 |
| 30 | 30.10 | 2.97 | 0.1 |
| 31 | 30.54 | 2.92 | 1.3 |
| 32 | 31.87 | 2.81 | 0.1 |
| 33 | 32.26 | 2.77 | 1.3 |
| 34 | 32.92 | 2.72 | 1.4 |
| 35 | 33.49 | 2.67 | 0.2 |
| 36 | 34.04 | 2.63 | 0.4 |
| 37 | 34.61 | 2.59 | 0.5 |
| 38 | 34.92 | 2.57 | 1.1 |
| 39 | 35.15 | 2.55 | 0.9 |
| 40 | 35.72 | 2.51 | 0.3 |
| 41 | 36.21 | 2.48 | 0.4 |
| 42 | 36.69 | 2.45 | 0.5 |
| 43 | 37.57 | 2.39 | 1.2 |
| 44 | 38.02 | 2.36 | 0.1 |
| 45 | 38.43 | 2.34 | 0.8 |
| 46 | 38.67 | 2.33 | 0.2 |
| 47 | 39.52 | 2.28 | 0.3 |
| 48 | 39.76 | 2.27 | 0.9 |

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A pharmaceutical composition, comprising:
a plurality of coated particles, each coated particle comprising:
a core particle comprising a hydrocortisone derivative selected from 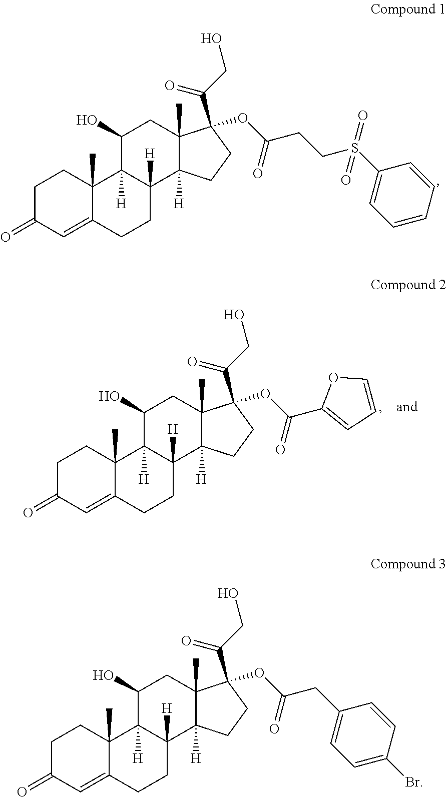
and a mucus penetration-enhancing coating surrounding the core particle, wherein the mucus penetration-enhancing coating comprises a
surface-altering agent comprising one or more of the following components:
a) a triblock copolymer comprising a hydrophilic block-hydrophobic block-hydrophilic block configuration, wherein the hydrophobic block has a molecular weight of at least about 2 kDa, and the hydrophilic blocks constitute at least about 15 wt % of the triblock copolymer, wherein the hydrophobic block associates with the surface of the core particle, and wherein the hydrophilic block is present at the surface of the coated particle and renders the coated particle hydrophilic,
b) a synthetic polymer having pendant hydroxyl groups on the backbone of the polymer, the polymer having a molecular weight of at least about 1 kDa and less than or equal to about 1000 kDa, wherein the polymer is at least about 30% hydrolyzed and less than about 95% hydrolyzed, or
c) a polysorbate,
wherein the surface altering agent is present on the outer surface of the core particle at a density of at least 0.01 molecules/nm²,
wherein the surface altering agent is present in the pharmaceutical composition in an amount of between about 0.001% to about 5% by weight; and
one or more pharmaceutically acceptable carriers, additives, or diluents;
wherein the pharmaceutical composition is suitable for administration to an eye of a subject.

2. The pharmaceutical composition of claim 1,
wherein the coating on the core particle is present in a sufficient amount to increase the concentration of the hydrocortisone derivative in an ocular tissue after administration when administered to the eye, compared to the concentration of the hydrocortisone derivative in the ocular tissue when administered as a core particle without the coating.

3.-5. (canceled)

6. The pharmaceutical composition of claim 1, wherein the hydrocortisone derivative is

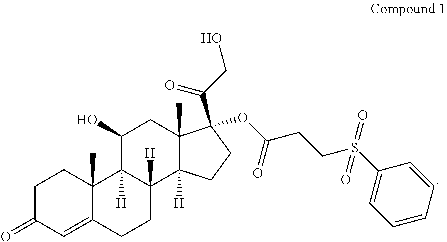

7. The pharmaceutical composition of claim 6, wherein Compound 1 is in crystalline form B having X-ray powder diffraction (XRPD) peaks at 5.88, 10.36, 13.18, 14.40, 15.55, 17.57, and 20.82±0.2°2θ.

8. The pharmaceutical composition of claim 1, wherein the hydrocortisone derivative is

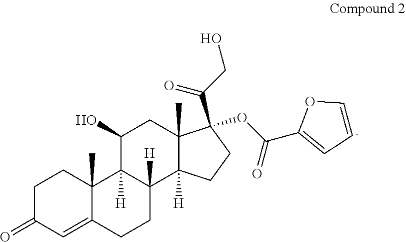

9. The pharmaceutical composition of claim 8, wherein Compound 2 is in crystalline form A having XRPD peaks at 5.83, 10.09, 11.72, 14.49, 15.32, and 15.66±0.2° 2θ.

10. The pharmaceutical composition of claim 1, wherein the hydrocortisone derivative is

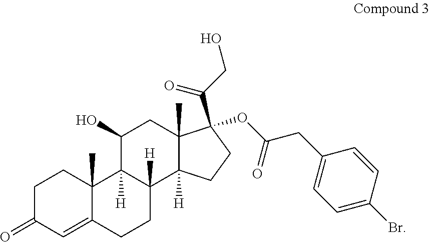

11. The pharmaceutical composition of claim 10, wherein Compound 3 is in crystalline form A having XRPD peaks at 5.08, 7.18, 13.90, and 20.45±0.2° 2θ.

12. The pharmaceutical composition of claim 10, wherein Compound 3 is in crystalline form B having XRPD peaks at 8.88, 12.66, 14.34, 19.02, 20.28, 20.63 and 25.71±0.2° 2θ.

13. The pharmaceutical composition of claim 1, wherein the surface-altering agent is present on the surfaces of the coated particles at a density of at least about 0.1 molecules per nanometer squared.

14. The pharmaceutical composition of claim 1, wherein the surface-altering agent is covalently attached to the core particles.

15. The pharmaceutical composition of claim 1, wherein the surface-altering agent is non-covalently adsorbed to the core particles.

16. The pharmaceutical composition of claim 1, wherein the surface-altering agent comprises the triblock copolymer.

17. The pharmaceutical composition of claim 16, wherein the hydrophilic blocks of the triblock copolymer constitute at least about 30 wt % of the triblock polymer and less than or equal to about 80 wt % of the triblock copolymer.

18. The pharmaceutical composition of claim 17, wherein the hydrophobic block portion of the triblock copolymer has a molecular weight of about 3 kDa to about 8 kDa.

19. The pharmaceutical composition of claim 17, wherein the triblock copolymer is poly(ethylene oxide)-poly(propylene oxide)-poly(ethylene oxide).

20. The pharmaceutical composition of claim 1, wherein the surface-altering agent has a molecular weight of at least about 4 kDa.

21. The pharmaceutical composition of claim 1, wherein the surface-altering agent comprises a linear polymer having pendant hydroxyl groups on the backbone of the polymer.

22. The pharmaceutical composition of claim 21, wherein the surface altering agent is poly(vinyl alcohol).

23. The pharmaceutical composition of claim 22, wherein the poly(vinyl alcohol) is about 70% to about 94% hydrolyzed.

24. The pharmaceutical composition of claim 1, wherein the hydrocortisone derivative is crystalline.

25. The pharmaceutical composition of claim 1, wherein the hydrocortisone derivative is amorphous.

26. The pharmaceutical composition of claim 1, wherein the hydrocortisone derivative is encapsulated in a polymer, a lipid, a protein, or a combination thereof.

27. The pharmaceutical composition of claim 1, wherein the hydrocortisone derivative comprises at least about 80 wt % of the core particle.

28. The pharmaceutical composition of claim 1, wherein the coated particles have an average size of about 10 nm to about 1 μm.

29. The pharmaceutical composition of claim 1, comprising one or more degradants of the hydrocortisone derivative, and wherein the concentration of each degradant is 0.1 wt % or less relative to the weight of the hydrocortisone derivative.

30. The pharmaceutical composition of claim 1, wherein the polydispersity index of the composition is less than or equal to about 0.5.

31. The pharmaceutical composition of claim 1, wherein the pharmaceutical composition is suitable for topical administration to the eye.

32. The pharmaceutical composition of claim 1, wherein the pharmaceutical composition is suitable for direct injection into the eye.

33. The pharmaceutical composition of any claim 1, wherein the one or more ophthalmically acceptable carriers, additives, or diluents comprises glycerin.

34. A method of treating, diagnosing, preventing, or managing an ocular condition in a subject, the method comprising: administering a pharmaceutical composition of claim 1 to an eye of a subject and thereby delivering the hydrocortisone derivative to a tissue in the eye of the subject.

35. The method of claim 34, comprising sustaining an ophthalmically efficacious level of the hydrocortisone derivative and/or its hydrocortisone metabolite in a palpebral conjunctiva, a fornix conjunctiva, a bulbar conjunctiva, or a cornea for at least 12 hours after administration.

36. The method of claim 34, comprising delivering the hydrocortisone derivative and/or its hydrocortisone metabolite to a tissue in the front of the eye of the subject.

37. The method of claim 34, comprising delivering the hydrocortisone derivative and/or its hydrocortisone metabolite to a tissue in the back of the eye of the subject.

38. The method of claim 37, wherein the tissue is a retina, a macula, a sclera, a cornea, a lid, aqueous humor, or a choroid.

39. The method of claim 34, wherein the ocular condition is inflammation, macular degeneration, macular edema, uveitis, glaucoma, or dry eye.

40. The pharmaceutical composition of claim 28, wherein the average particle size is measured by dynamic light scattering.

41. The pharmaceutical composition of claim 30, wherein the polydispersity index is measured by dynamic light scattering.

42. The pharmaceutical composition of claim 2, wherein the ocular tissue is an anterior ocular tissue.

43. The pharmaceutical composition of claim 42, wherein the anterior ocular tissue is a palpebral conjunctiva, a bulbar conjunctiva, a fornix conjunctiva, an aqueous humor, an anterior sclera, a cornea, an iris, or a ciliary body.

44. The pharmaceutical composition of claim 2, wherein the ocular tissue is a tissue at the back of the eye.

45. The pharmaceutical composition of claim 42, wherein the anterior ocular tissue is a vitreous humor, a vitreous chamber, a retina, a macula, a choroid, a posterior sclera, a uvea, an optic nerve, or the blood vessels or nerves which vascularize or innervate a posterior ocular region or site of they eye.
